SEQUENCE LISTING (1 sequence)

SEQ ID NO: 1 - protein, 9 aa, Unknown
  Description of Unknown “LAGLIDADG” family peptide motif sequence
LAGLIDADG

(19) United States
(12) Patent Application Publication
(10) Pub. No.: US 2019/0093117 A1
(43) Pub. Date: Mar. 28, 2019

(54) METHODS AND COMPOSITIONS FOR VIRAL-BASED GENE EDITING IN PLANTS

(71) Applicant: R. J. Reynolds Tobacco Company, Winston-Salem, NC (US)

(72) Inventors: Xingpeng Li, Lewisville, NC (US); Yijian He, Greensboro, NC (US); Gregory P. Pogue, Austin, TX (US); Emmett Ernest Hiatt, East Bend, NC (US); Darlene Madeline Lawson, Kernersville, NC (US)

(21) Appl. No.: 16/048,585

(22) Filed: Jul. 30, 2018

Related U.S. Application Data
(60) Provisional application No. 62/539,160, filed on Jul. 31, 2017.

(51) Int. Cl. C12N 15/82; C12N 9/22; C12N 15/11

(52) U.S. Cl. CPC C12N 15/8213; C12N 9/22; C12N 15/11; C12N 2770/00043; C12N 2310/20; C12N 2770/00021; C12N 15/8203

(57) ABSTRACT

The present disclosure provides compositions and methods for editing a target site of a plant genome by delivery of functional editing components using modified tobacco mosaic virus (mTMV). The methods disclosed herein can be used to deliver a gene editing system, such as a DNA endonuclease, to a tobacco plant cell for modification of a target site of the plant genome. Further, the methods and compositions disclosed herein provide for production of a RNA molecule encoding a meganuclease in vitro prior to delivery of the RNA to a plant cell. After introduction of the nucleic acid molecule encoding a functional editing component and subsequent expression of the functional editing components, the plant can be cultured and allowed to produce seeds having an edit at a genomic target site. The seeds can then undergo embryo rescue and be cultured to produce a modified plant without heterologous genetic material.

### RELATED APPLICATIONS

This application claims priority to U.S. Provisional Patent Application No. 62/539,160, entitled “Methods and Compositions For Viral-Based Gene Editing In Plants,” filed Jul. 31, 2017. The entire contents of U.S. Provisional Patent Application No. 62/539,160 is incorporated by reference in its entirety herein.

### FIELD OF THE INVENTION

The present disclosure provides compositions and methods for editing a target site of a plant genome by delivery of gene editing components.

### BACKGROUND

Transgenic technologies provide powerful tools for modifying plants. Still, the application of transgenic modification to introduce foreign DNA into the plant genome has been associated with public safety concerns. There is a need for the application of techniques which can be used to modify the plant genome, but that do not introduce foreign genetic material. Thus, there is a need to develop systems to improve plant traits without introducing foreign DNA into the plant. Particularly, there is a need for tools that can produce a site-directed modification of a plant genome without integration of foreign DNA.

Transient expression of recombinant proteins in Nicotiana plants is a rapid and convenient alternative to stable transformation because of the dramatic increase in speed and yield offered. (Fischer et al., Curr Opin Plant Biol 2004; 7(2):152-8). There are two basic types of transient expression systems, depending on the expression vectors used. The first type is based on standard (non-viral) vectors having the coding sequence of interest under transcriptional control of strong constitutive promoters. The second type of transient expression utilizes plant viruses, predominantly RNA viruses, adapted as expression vectors. With respect to transient expression systems, the virus-based systems can be divided in two sub-groups: vectors built on the basis of independently functioning (replication, local and systemic movement in planta) virus vectors and vectors built on virus vectors containing minimal genes allowing replication and local movement but not supporting systemic infections. Production using independent virus vectors can be initiated by inoculation of plants with in vitro synthesized infectious RNA transcripts encoding the vector, while minimal virus vectors are generally launched using Agrobacterium tumefaciens transfection, T-DNA transfer and subsequent transcription of the infectious RNA encoding the vector in planta. The common element in these expression systems is the use of Nicotiana benthamiana (Nb) as the preferred production host.

The isolation, cloning, transfer and recombination of DNA segments, including coding sequences and non-coding sequences, can be carried out using restriction endonuclease enzymes. Although several approaches have been developed to target a specific site for modification in the genome of a plant, there still remains a need for more efficient and effective methods for producing a fertile plant having an altered genome comprising specific modifications in a defined region of the genome of the plant.

### SUMMARY OF THE DISCLOSURE

The term embodiment and like terms are intended to refer broadly to all of the subject matter of this disclosure and the claims below. Statements containing these terms should be understood not to limit the subject matter described herein or to limit the meaning or scope of the claims below. This summary is a high-level overview of various aspects of the disclosure and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this disclosure, any or all drawings and each claim

The present disclosure provides compositions and methods for editing a target site of a plant genome by delivery of gene editing components using modified tobacco mosaic virus (mTMV). In particular, the present disclosure relates to methods for modification of the tobacco genome to produce modified tobacco material for use in products made or derived from tobacco, or that otherwise incorporate tobacco, and are intended for human consumption. The disclosure may be embodied in a variety of ways.

In certain embodiments, the present disclosure provides compositions and methods for editing a target site of a plant genome by delivery of functional editing components using a modified tobacco mosaic virus (mTMV) and using no DNA intermediate or exogenous gene insertion into the plant genome. The methods disclosed herein can be used to deliver a gene editing system, such as a DNA endonuclease, to a plant cell for modification of a target site of the plant genome, such as the genome of a tobacco cell. Further, the methods and compositions disclosed herein provide for production of an RNA molecule encoding a meganuclease in vitro prior to delivery of the RNA to a plant cell. After introduction of the nucleic acid molecule encoding a functional editing component and subsequent expression of the functional editing components, the plant can be cultured and allowed to produce seeds having an edit at a genomic target site. The seeds can then undergo embryo rescue and cultured to produce a modified plant without heterologous genetic material.

For example, in certain embodiments disclosed is a method for modifying a target site in the genome of a tobacco plant cell, the method comprising: introducing a nucleic acid encoding a functional editing component into the tobacco plant cell, wherein the functional editing component introduces a modification at the target site in the genome of the tobacco plant cell. In some embodiments, the nucleic acid is an RNA molecule. In certain embodiments, the functional editing component encodes an endonuclease that cleaves DNA. The endonuclease may be one of a meganuclease and/or a guide RNA and/or Cas9 endonuclease. In some embodiments, the nucleic acid comprises an RNA expression vector. For example, in some embodiments the vector is a tobacco mosaic virus (TMV) vector. Thus, in one aspect a method is provided for modifying a target site in the genome of a plant cell, the method comprising: introducing a tobacco mosaic virus (TMV) genome modified to comprise an RNA molecule comprising a nucleic acid sequence encoding a DNA endonuclease specific for a target site, wherein, when expressed, the DNA endonuclease introduces a modification at the target site.

The functional editing component may be operably linked to a promoter. In certain embodiments, the promoter is one that is functional in a plant cell. For example, in some embodiments, the promoter is one of a CaMV35S, a T7 RNA polymerase promoter, or a coat protein subgenomic promoter. Additionally and/or alternatively, the nucleic acid may be synthesized in the plant cell or may be synthesized in vitro prior to introducing the nucleic acid nucleic acid encoding a functional editing component into the plant cell.

In specific embodiments, the target site is in a gene encoding a PDS (Phytoene desaturase), nicotine synthase, or a nicotine demethylase. Or another gene may be targeted.

The nucleic acid molecule can be a RNA molecule encoding the DNA endonuclease. The modification of the target site can be at least one deletion, insertion or substitution of one or more nucleotides in the target site. The modification can also be a double strand break. For example, in some aspects, the DNA endonuclease is a meganuclease. For example, the meganuclease can be a meganuclease modified (e.g., genetically engineered) to be specific for the target site in a gene encoding a PDS (Phytoene desaturase), nicotine synthase, or a nicotine demethylase. The modified TMV genome can further comprise a promoter operably linked to the nucleic acid sequence encoding an endonuclease, wherein the promoter is active in the plant cell.

As noted above, in some aspects, the RNA molecule is located on a vector. In specific aspects the nucleic acid molecule encoding a DNA endonuclease is located in a GENEWARE® TMV-based gene expression vector such as pDN15, pBS1057, p30B or other specific vector system. The nucleic acid molecule, such as a RNA molecule, can be synthesized in vitro prior to delivery to a plant cell. The RNA molecule can be introduced by mechanical transmission using rubbing, high-pressure spray, gene gun, or similar technologies. In some cases, the nucleic acid encoding the functional editing component is mechanically introduced to the plant cell by rubbing, high pressure spray, or using a gene gun.

The methods disclosed herein also comprise propagating plants having the modified target site and plants and seeds made by the methods described herein and having the modified target site. In some cases the plant and/or seed is N. tabacum tobacco or an N. rustica tobacco. Or, the plants and/or seeds may be other tobaccos disclosed herein.

For example, the method may further comprise removing a part of the plant comprising the nucleic acid encoding the functional editing component and culturing the part of the plant on selection medium. In some cases the plant part comprising the nucleic acid encoding the functional editing component is removed from the leaf, meristem, shoot, and/or flower of the plant.

In some aspects, the method further includes culturing the plant part to produce a regenerated plant. The method may also comprise confirming the modification of the target site in the plant part and/or plant derived therefrom. Additionally and/or alternatively, the method may comprise isolating at least one plant cell comprising a modification at the target site. The at least one cell and/or plant part may be cultured to produce a plant having the modification at the target site, and in some embodiments, the plant can be cultured until the plant produces seeds comprising a modification at the target site of the genome. In some embodiments, embryo rescue can be performed on the seeds which comprise a modification at the target site of the genome. Tobacco seed produced from the plant or second plant comprising the modification at the target site can be planted and cultured to produce a plant having the modification at the target site. The plant may then be harvested, and used to produce a tobacco product.

Plants, such as tobacco plants, are also provided that are produced by the methods disclosed herein. Seeds, such as tobacco seeds, are provided that are produced by the methods disclosed herein.

In some aspects, a tobacco mosaic virus (TMV) genome is provided that is modified to comprise a nucleic acid sequence encoding a DNA endonuclease, such as a meganuclease. In some embodiments, the vector may be a GENEWARE® vector. In some embodiments, the meganuclease is specific for a target site in a gene encoding a PDS (Phytoene desaturase), nicotine synthase, or a nicotine demethylase. Or the meganuclease may be specific for other targets. The modified TMV (mTMV) genome can comprise a promoter, such as the CaMV35S promoter, a T7 RNA polymerase promoter, or a coat protein subgenomic promoter operably linked to the nucleic acid sequence encoding a DNA endonuclease. A modified tobacco mosaic virus is provided comprising the TMV genome disclosed herein. Also disclosed are vectors comprising these constructs and additional embodiments disclosed herein.

Also disclosed are a tobacco plant, tobacco plant part, or tobacco plant cell comprising an RNA expression vector comprising a nucleic acid sequence encoding a functional editing component. In certain embodiments, the functional editing component is an endonuclease that cleaves DNA at a target site. The endonuclease may be one of a meganuclease and/or a guide RNA and/or Cas9 endonuclease. In some embodiments, the nucleic acid comprises an RNA expression vector. For example, in some embodiments the vector is a tobacco mosaic virus (TMV) vector. The functional editing component may be operably linked to a promoter. In certain embodiments, the promoter is one that is functional in a plant cell. For example, in some embodiments, the promoter is one of a CaMV35S, a T7 RNA polymerase promoter, or a coat protein subgenomic promoter. In specific embodiments, the target site is in a gene encoding a PDS (Phytoene desaturase), nicotine synthase, or a nicotine demethylase. Or another gene may be targeted.

Also in certain embodiments, provided are tobacco plants, a tobacco plant part, or a tobacco plant cell and/or tobacco seeds made by modifying a target site in the genome of a tobacco plant cell by introducing an RNA molecule comprising a nucleic acid sequence encoding a DNA endonuclease into the tobacco plant cell, wherein, when expressed, the DNA endonuclease introduces a modification at the target site in the genome of the tobacco plant cell. In an embodiment, the vector may be a GENEWARE® vector. In some embodiments, the nuclease may be a meganuclease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The disclosure may be better understood by reference to the non-limiting description of the drawings. In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

FIG. 1 shows a map the RJRTARL002 vector comprising the GENEWARE® pDN15 vector modified to express the cycle 3 green fluorescent protein (c3GFP) according to an embodiment of the disclosure.

FIG. 2 shows an example of three different types of tobacco, Nicotiana tabacum var. Xanthi, Nicotiana benthamiana, and Nicotiana tabacum var. K326 seven days post-inoculation with the RJRTARL002 vector according to an embodiment of the disclosure. The images in the top row are exposed to UV light allowing for visualization of the green fluorescent protein. The images in the bottom row are exposed to white light allowing for visualization of the area of infection.

### DETAILED DESCRIPTION

The present disclosure now will be described more fully hereinafter. The disclosure may be embodied in many different forms and should not be construed as limited to the aspects set forth herein; rather, these aspects are provided so that this disclosure will satisfy applicable legal requirements. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

When introducing elements of the present disclosure or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. It is understood that aspects and embodiments of the disclosure described herein include “consisting” and/or “consisting essentially of” aspects and embodiments.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

Various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terms “target site”, “target sequence”, “target DNA”, “target locus”, “genomic target site”, “genomic target sequence”, and “genomic target locus” are used interchangeably herein and refer to a polynucleotide sequence in the genome (including choloroplastic and mitochondrial DNA) of a plant cell which is recognized by a functional editing component. The target site can be an endogenous site in the plant genome, or alternatively, the target site can be heterologous to the plant and thereby not be naturally occurring in the genome, or the target site can be found in a heterologous genomic location compared to where it occurs in nature. As used herein, terms “endogenous target sequence” and “native target sequence” are used interchangeable herein to refer to a target sequence that is endogenous or native to the genome of a plant and is at the endogenous or native position of that target sequence in the genome of the plant.

As used herein, a “functional editing component” refers to a polynucleotide comprising a coding portion that encodes a component of a gene-editing system. In some cases, the functional editing component is an endonuclease. In some cases, the functional editing component is a gRNA and to which a RNA-guided endonuclease (e.g., an endonuclease such as endonuclease A or a Cas9 endonuclease) can be targeted. A RNA-guided endonuclease can then induce a double-strand break in the plant cell genome at the target site. Or, other functional editing molecules may be used.

As used herein, a “genome-editing endonuclease” is a type or component of a gene-editing system. Such gene editing systems are used to modify genomic DNA to generate “genome-edited” plants such as those described herein. Such modifications do not include incorporation of foreign DNA, but do include repair of DNA by the plants own repair system. For example, a functional editing component can include a polynucleotide RNA that encodes a genome-editing DNA endonuclease that can be transferred to plant cell. Such polynucleotide RNA can be inserted in a RNA virus backbone. Such endonucleases include a meganuclease, a guide RNA, and CRISPR-cas9, or portion thereof, or a polynucleotide that encodes a guided endonuclease, such as TALEN, ZFN, CRISPR-cas9, or meganuclease (homing endonuclease). In particular embodiments, functional editing components are operably linked to sufficient regulatory elements such as a promoter, so that expression is achieved in the plant cell of interest.

As used herein, meganucleases are endodeoxyribonucleases (i.e., endonucleases) characterized by a large recognition site (double-stranded DNA sequences of 12 to 40 base pairs); as a result this site generally occurs only once in any given genome. Meganucleases may be used to modify all genome types, whether bacterial, plant or animal.

As used herein, “homologous recombination” (HR) includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombination events: the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al., (1982) Cell 31:25-33; Shen and Huang, (1986) Genetics 112:441-57; Watt et al., (1985) Proc. Natl. Acad. Sci. USA 82:4768-72, Sugawara and Haber, (1992) Mol Cell Biol 12:563-75, Rubnitz and Subramani, (1984) Mol Cell Biol 4:2253-8; Ayares et al., (1986) Proc. Natl. Acad. Sci. USA 83:5199-203; Liskay et al., (1987) Genetics 115:161-7.

A “modified nucleotide” or “edited nucleotide” refers to a nucleotide sequence of interest that comprises at least one alteration when compared to its non-modified nucleotide sequence. Such “alterations” include, for example: substitution of at least one nucleotide, a deletion of at least one nucleotide, an insertion of at least one nucleotide, or any combination thereof.

The term “plant” refers to whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. Plant parts include differentiated and undifferentiated tissues including, but not limited to roots, stems, shoots, leaves, pollens, seeds, tumor tissue and various forms of cells and culture (e.g., single cells, protoplasts, embryos, and callus tissue). The plant tissue may be in plant or in a plant organ, tissue or cell culture. The term “plant organ” refers to plant tissue or a group of tissues that constitute a morphologically and functionally distinct part of a plant. The term “genome” refers to the entire complement of genetic material (genes and non-coding sequences) that is present in each cell of an organism, or virus or organelle; and/or a complete set of chromosomes inherited as a (haploid) unit from one parent. “Progeny” comprises any subsequent generation of a plant.

A genome-edited plant includes, for example, a plant which comprises within its genome a heterologous polynucleotide introduced by deletion of a nucleotide or plurality of nucleotides in a genomic sequence. The genomic sequence may in some cases be DNA. Additionally and/or alternatively, the deletion may be in RNA encoded by the DNA. The deletion may be stably integrated within the genome such that the modified nucleotide sequence is passed on to successive generations. A genome-edited plant can also comprise more than one modification within its genome. Each modification (e.g., deletion, substitution) may confer a different trait to the genome-edited plant. The edited genome can include any cell, cell line, callus, tissue, plant part or plant, the genotype of which has been altered by the presence of the modified nucleic acid including those plants initially so altered as well as those created by sexual crosses or asexual propagation from the initial transgenic. The alterations of the genome (chromosomal or extra-chromosomal) by conventional plant breeding methods, by the genome editing procedure described herein that results in an insertion of a foreign polynucleotide, or by naturally occurring events such as random cross-fertilization, non-recombinant viral infection, non-recombinant bacterial transformation, non-recombinant transposition, or spontaneous mutation are not intended to be regarded as genome-edited plants. In specific embodiments, a genome-edited plant comprises a mutation (deletion, insertion or substitution) at a genomic locus, but does not have any heterologous DNA inserted into the plant genome. For example, a genome-edited plant can be created by cleavage of a target site in the genome of the plant and subsequent repair by non-homologous end joining (NHEJ) that introduces a mutation during the repair process.

Overview

The disclosure provides compositions and methods for genome editing of plants. Methods and compositions described herein may utilize one or more vectors to deliver nucleic acid molecules for the modification of a target site in a plant genome. In certain embodiments, the nucleic acid included in the vector that produces a DNA endonuclease. In some embodiments, the vectors are based on a viral vector platform, such as the TMV vector for delivery of nucleic acid molecules to plants or expression of the nucleic acid molecule. In some embodiments, the endonuclease is a genome editing endonuclease.

Virus Vectors

Plant virus-based vectors can allow for the rapid, transient expression of proteins and nucleic acids in whole plants. Although many different plant viruses have been modified to function as expression vectors, Tobacco Mosaic Virus (TMV) based vectors can express consistently high levels of foreign proteins or nucleic acids in plants and were among the first viral vectors to be used for either gene expression or gene silencing in plants (Fitzmaurice et al. 2002; Pogue et al., Ann Rev Phytopathol 2002; 40:45-74.). TMV has a positive sense single stranded RNA genome of approximately 6400 nucleotides. TMV virions are rigid rod shaped particles composed of approximately 2100 copies of the 17.5 kDa coat protein (CP), helically encapsidating the genomic RNA. The viral proteins involved in RNA replication are directly transcribed from the genomic RNA, whereas expression of internal genes is through the production of subgenomic RNAs. The production of subgenomic RNAs is controlled by sequences in the TMV genome, which function as subgenomic promoters. The CP is translated from a subgenomic RNA and is the most abundant protein and RNA produced in the infected cell. In a TMV infected plant there are several milligrams of CP produced per gram of infected tissue.

GENEWARE® expression vectors take advantage of both the strength and duration of the TMV CP promoter's activity to reprogram the translational priorities of the plant host cells so that virus-encoded proteins are synthesized at similar high levels as the TMV coat protein (Pogue et al., 2002.; Shivprasad et al., Virology 1999; 255(2):312-23).

In other systems, full-length cDNA copies of the TMV RNA genome under the control of the T7 RNA polymerase promoter have been constructed in an E. coli compatible plasmid. Manipulations to the virus cDNA can be performed using standard recombinant DNA procedures and the recombinant DNA transcribed in vitro with T7 RNA polymerase to generate infectious RNA. The infectious transcripts are used to infect primarily Nb plants. The infectious RNA enters plant cells via wounds induced by an applied abrasive (Pogue et al. 1998). The virus replicates in the initial cell, moves to adjacent cells to produce round infection foci and then enters the plant's vascular system for transport to aerial leaves. There it systematically infects the majority of cells in each infected leaf. The foreign gene is expressed in all cells that express other virus protein products, including the replicase, movement protein (MP) and CP. The TMV expression system has been used to produce over 200 recombinant proteins and antigens, including human enzymes, antimicrobials, cytokines, vaccines and immunoglobulin fragments. In addition, the use of RNAi, antisense, and hairpin loop constructs have been used to “silence” thousands of endogenous genes (Fitzmaurice et al., 2002; Kentucky Bioprocessing Co. unpublished data).

One advanced transient minimal virus-based system launched by infiltration of plants with Agrobacterium strains is based on the tobamovirus and potexvirus transient systems. The technology and its applications have been described in numerous publications (Pogue et al., 2010. Gleba & Giritch in Recent Advances in Plant Virology. eds., Caranta, Tepfer, & Lopez-Moya. Norfolk, Caister Academic Press 2011:387-412). These expression vectors are generally single component (although two component systems that recombine in vivo to form fully functional genomes have been used as well), with full genomes expressed from an operatively attached DNA-dependent, RNA-polymerase II promoter, such as from cauliflower mosaic virus 35S RNA cistron. These expression vectors have proven versatile with demonstrated expression of numerous heterologous proteins, including cytokines, interferon, bacterial and viral antigens, growth hormone, vaccine antigens, single chain antibodies and monoclonal antibodies (mAbs). These expression levels support economically viable production of products ranging from pharmaceutical and diagnostic analytes, to tissue culture excipients and biochemical reagents of 5-150 kDa molecular weight.

These vectors can be built from two different plant virus genomes: TMV-related virus turnip vein clearing tobamovirus (TVCV), with appropriately added introns and removal of cryptic intron processing sites, or potato virus X (PVX). The cDNAs of the viral replicons, encoding all the genes required for virus RNA replication, are launched via Agro-infiltration process that initially introduces the virus vectors, carried by the introduced Agrobacterium, to many cells throughout the transfected plant. The vector then is “activated” by transcription from the T-DNA region to produce the virus RNA in vivo and transits it to the cytoplasm for RNA amplification via virus-encoded proteins. Most vectors encode requisite proteins for cell to cell movement, including the movement (30 K) protein from tobamovirus-based vectors and the triple block products and coat protein for potexvirus-based vectors. These proteins allow movement of the virus vector genome locally within an inoculated leaf resulting in the majority of cells being infected and becoming production sites for the desired protein product in as few as 5-7 days. Aerial parts of the plant are harvested generally by 6-8 days post inoculation (dpi) and extracted for the desired product.

An alternative strategy for gene expression in plants involves transient or stable plant transformation. Recombinant DNA technology has made it possible to insert foreign DNA sequences into the genome of a plant (transformation), thus, altering the plant's phenotype to generate a transgenic plant. The most commonly used plant transformation methods used for recombinant modification are Agrobacterium infection and biolistic particle bombardment in which transgenes integrate into a plant genome in a random fashion and in an unpredictable copy number. Thus, efforts are undertaken to control transgene integration in plants to provide more targeted integration for better prediction of the resulting phenotype.

Some plant viruses have segmented genomes, in which two or more physically separate pieces of nucleic acid together make up the viral genome. In particular cases, these separate pieces are packaged together in the same viral capsid; in other viruses (i.e., those with multipartite genomes), each genome segment is packaged into its own viral particle. Infection of a plant by a viral genome can typically be accomplished by delivery either of plant viral nucleic acid (e.g., RNA) or capsid containing the packaged genome. In order to enter and infect a plant cell, plant viruses need to cross the cell wall, in addition to protective layers of waxes and pectins. Most or all plant viruses are thought to rely on mechanical breach of the cell wall, rather than on cell-wall-surface receptors, to enter a cell. Such a breach can be caused, for example, by physical damage to the cell, by an organism such as a bacterium, a fungus, a nematode, an insect, or a mite that can deliver the virus. In the laboratory, viruses are typically administered to plant cells simply by rubbing the virus on the plant.

Once the virus has entered (infected) a cell, it typically replicates within the infected cell and then spreads locally. For example, the virus can replicate and spread from cell to cell within leaves that were initially infected. Following local spread, the virus may move into uninfected leaves, e.g., upper leaves of the plant, which is referred to as systemic infection or systemic spread. In general, cell-to-cell spread of many plant viruses requires a functional movement protein while systemic spread requires a functional coat protein (and, generally, also a functional movement protein). In addition to functional movement and coat protein encoding components, viruses may contain additional components that are either required for local or systemic spread or facilitate such spread. These cis-acting components may be either coding or noncoding components. For example, they may correspond to portions of a 3′ untranslated region (UTR, also referred to as NTR) of a viral transcript (i.e., they may provide a template for transcription of a 3′ untranslated region of a viral transcript). Thus important viral components for infection can be either coding or noncoding regions of a viral genome.

In order to successfully establish either a local (intraleaf) or systemic infection a virus must be able to replicate. Many viruses contain genes encoding one or more proteins that participate in the replication process (referred to herein as replication proteins or replicase proteins). For example, many RNA plant viruses encode a RNA polymerase. Additional proteins may also be required (e.g., helicase or methyltransferase protein(s)). The viral genome may contain various sequence components in addition to functional genes encoding replication proteins, which are also required for or facilitate replication. Local intraleaf infections require the virus to move cell-to-cell as mediated by movement facilitating protein(s). For example in the case of TMV, the MP protein expression is required for intraleaf infections. The CP is not required. Other viruses, such as potatovirus potexvirus, require movement proteins and CP expression to move from cell-to-cell and establish an intraleaf infection.

Any virus that infects plants may be used to prepare a viral vector or vector system for gene editing as disclosed herein. For example, the genome of any virus that infects plants may be modified to express a functional editing component in order to edit a target site of the genome of the infected plant. As discussed in detail below, in certain embodiments the GENEWARE® vector is used.

In particular embodiments, viruses used in the methods and compositions disclosed herein may be ssRNA viruses, and specifically, ssRNA viruses with a (+)-stranded genome. Techniques and reagents for manipulating the genetic material present in such viruses are known in the art. For example, a DNA copy of the viral genome may be prepared and cloned into an expression vector, particularly a bacterial vector or a Ti plasmid. Certain ssDNA viruses, including particularly geminiviruses, can also be used to deliver functional editing components to plant cells. It will be appreciated that in general the vectors and viral genomes of the invention may exist in RNA or DNA form. In addition, where reference is made to a feature such as a genome or portion thereof of a RNA virus, which is present within a DNA vector, it is to be understood that the feature is present as the DNA copy of the RNA form. This cDNA is converted into infectious RNA transcripts through transcription in vitro using T7 or other polymerase or in vivo using host DNA dependent RNA polymerase II using the Agrobacterium or particle delivered Ti DNA as template.

Viruses of a number of different types may be used in accordance with the gene editing methods and compositions disclosed herein. Exemplary viruses include members of the Bromoviridae (e.g., bromoviruses, alfamoviruses, ilarviruses) and Tobamoviridae. Certain virus species include, for example, Alfalfa Mosaic Virus (AlMV), Apple Chlorotic Leaf Spot Virus, Apple Stem Grooving Virus, Barley Stripe Mosiac Virus, Barley Yellow Dwarf Virus, Beet Yellow Virus, Broad Bean Mottle Virus, Broad Bean Wilt Virus, Brome Mosaic Virus (BMV), Carnation Latent Virus, Carnation Mottle Virus, Carnation Ringspot Virus, Carrot Mottle Virus, Cassava Latent Virus (CL V), Cowpea Chlorotic Mottle Virus, Cowpea Mosaic Virus (CPMV), Cucumber Green Mottle Mosaic Virus, Cucumber Mosaic Virus, Lettuce Infectious Yellow Virus, Maize Chlorotic Mottle Virus, Maize Rayado Fino Virus, Maize Streak Virus (MSV), Parsnip Yellow Fleck Virus, Pea Enation Mosaic Virus, Potato Virus X, Potato Virus Y, Raspberry Bushy Dwarf Virus, Rice Necrosis Virus (RNV), Rice Stripe Virus, Rice Tungro Spherical Virus, Ryegrass Mosaic Virus, Soilborne Wheat Mosaic Virus, Southern Bean Mosaic Virus, Tobacco Etch Virus (TEV), Tobacco Mosaic Virus (TMV), Tobacco Necrosis Virus, Tobacco Rattle Virus, Tobacco Ring Spot Virus, Tomato Bushy Stunt Virus, Tomato Golden Mosaic Virus (TGMV), and Turnip Yellow Mosaic Virus (TYMV). In specific embodiments the virus is a potyvirus, cucomovirus, bromovirus, tobravirus, or potexvirus. In an embodiment, Tobacco Mosaic Virus (TMV) is used.

Elements of these plant viruses can be genetically engineered according to known techniques (see, for example, Sambrook et al., Molecular Cloning, 2nd Edition, Cold Spring Harbor Press, N Y, 1989; Clover et al., Molecular Cloning, IRL Press, Oxford, 1985; Dason et al., Virology, 172:285-292, 1989; Takamatsu et al., EMBO J6:307-311, 1987; French et al., Science 231: 1294-1297, 1986; Takamatsu et al., FEBS Lett. 269:73-76, 1990; Yusibov and Loesch-Fries, Virology, 208(1): 405-7, 1995. Spitsin et al., Proc Natl Acad Sci USA, 96(5): 2549-53, 1999, etc.) to generate viral vectors for use in accordance with the gene editing methods and compositions disclosed herein.

As noted above, in certain embodiments, the viral vectors used in the methods and compositions disclosed herein are TMV vectors modified to express the components of a gene editing system (functional editing components), such as a DNA endonuclease. As used herein a “TMV vector” is a DNA or RNA vector comprising at least one functional element of the TMV genome. TMV is a positive-sense single stranded RNA virus that infects a wide range of plants, especially tobacco and other members of the family Solanaceae. The TMV genome consists of a 6.3-6.5 kb single-stranded (ss) RNA. The 3′-terminus has a tRNA-like structure. The 5′ terminus has a methylated nucleotide cap (m7G5′pppG). The genome can encode 4 open reading frames (ORFs), two of which produce a single protein due to ribosomal readthrough of a leaky UAG stop codon. The 4 genes encode a replicase (with methyltransferase [MT] and RNA helicase [Hel] domains), a RNA-dependent RNA polymerase, a so-called movement protein (MP) and a capsid protein (CP).

As used herein, an element of the TMV genome or TMV genome element refers to at least one nucleic acid molecule (i.e., gene) encoding a functional protein necessary for TMV replication and/or TMV infection. For example, an element of the TMV genome refers to a gene encoding a functional replicase or portion thereof (e.g., MT or Hel domain), a RNA-dependent RNA polymerase, movement protein (MP), and/or capsid protein (CP). In some embodiments, the modified TMV (mTMV) genome comprises genes encoding a replicase, movement protein, and capsid protein without a RNA-dependent RNA polymerase.

In particular embodiments of the methods and compositions disclosed herein, a TMV vector comprises all elements of the TMV genome. In other embodiments, the TMV genome elements are divided among at least two separate vectors, such that a complete and functional TMV can assemble following expression of the TMV elements from each of the vectors. Thus, when at least two vectors are employed, one or both of the vectors are incapable of systemic infection alone, but together can provide all functions needed to support systemic TMV infection and allow expression of functional editing components for modification of a target site of a plant genome. Thus the methods and compositions disclosed herein provide the recognition that viral components can complement each other in trans, to provide systemic infection capability and/or expression of functional editing components for modification of a target site in a plant genome. In specific embodiments, the TMV vector is based on the U1 strain of TMV. For example, the TMV vector can be a GENEWARE® pDN15 vector. The GENEWARE® vector can be based on the pUC19 backbone. See, WO 99/36516, herein incorporated by reference.

In specific embodiments, the viral proteins involved in RNA replication are directly transcribed from the genomic RNA, whereas expression of internal genes occurs through the production of subgenomic RNAs. The production of subgenomic RNAs is controlled by RNA sequences in the TMV genome, which function as subgenomic promoters. The coat protein is translated from a subgenomic RNA and is the most abundant protein and RNA produced in the infected cell. In a TMV-infected plant there are several mg of coat protein produced per gram of infected tissue. Tobacco mosaic viral expression vectors take advantage of both the strength and duration of this strong subgenomic promoter's activity.

In certain embodiments, the vector comprises the GENEWARE® system. GENEWARE® vectors allow expression of foreign proteins or peptides by two distinct methods: 1) Independent gene expression: by adding a foreign gene for expression in place of the virus coat protein so it will be expressed from the endogenous virus coat protein promoter. For example, the nucleic acid sequence encoding a DNA endonuclease can be operably linked to the virus coat protein promoter. A second coat protein promoter of lesser transcriptional activity and non-identity in sequence is placed downstream of the heterologous coding region and a virus coat protein or selectable marker encoding gene may then be added. This encodes a third subgenomic RNA (including the MP expressing RNA) allowing the virus vector to express all requisite genes for virus replication and systemic movement in addition to the heterologous gene intended for overexpression. 2) Display of immunogenic peptides on the surface of virus particles: The TMV virion is a rigid rod of ^{˜}18 nm diameter and 300 nm length. The structure of the virion and coat protein has been determined by X-ray diffraction revealing a structure of approximately 2,130 coat protein subunits arranged in a right-handed helix encapsidating the genomic RNA, with 16.3 subunits per turn.

Functional Editing Components

The viral vector may be used to deliver a functional editing component to a plant cell or cells. Functional editing components can include any nucleic acid or amino acid that contributes to modification of the genome of a plant. In some embodiments, the methods disclosed herein can take advantage of the site specificity of certain endonucleases to cleave at least one recognition sequence in an endogenous polynucleotide of interest (e.g., endogenous gene of interest). Following cleavage, the site can be edited or have an exogenous gene of interest inserted in the target site. Any endonuclease that specifically or preferentially cleaves the corresponding recognition sequences can be used in the methods and compositions disclosed herein. By using endonucleases that specifically and preferentially cleave the recognition sequences and endogenous recognition sequences, cleavage at sites other than the recognition sequences is minimized and efficiency of cleavage is thereby increased. Accordingly, the endonucleases for cleavage of the recognition sequences and endogenous recognition sequences disclosed herein can be a meganuclease as for example, a meganuclease that functions as a genome-editing endonuclease, a zinc finger nuclease, a TALEN, a compact TALEN, a megaTAL, or a CRISPR.

Other forms of editing enzymes could be used in other embodiments. One such approach that has garnered attention is utilization of zinc-finger nucleases (ZFNs) (Antunes et al., BMC Biotechnology (2012), 12:86). ZFNs, chimeric fusions between a zinc-finger DNA binding domain and the FokI nuclease domain, have the ability to recognize and cut existing sites in a genome because the zinc-finger domain can be engineered to recognize a variety of different DNA sequences. Engineered ZFNs have been used to target homologous integration at native sites in the human genome. ZFNs have also been tested in Arabidopsis, tobacco, and maize and shown to be capable of targeting mutations to introduced sites by NHEJ and homologous recombination (HR) with frequencies as high as 16% and 2%, respectively. However, two potentially significant limitations of ZFN are reported: (1) toxicity in plants and mammalian cells, presumed to be caused by “off-site” cleavage, and (2) imprecise events associated with their cleavage (e.g., deletions, small insertions).

In addition, a similar approach to ZFNs has been obtained by fusing the FokI domain to transcription activator-like (TAL) effector proteins identified in plant pathogenic bacteria from the genus Xanthomonas. These TAL effector nucleases (TALEN) have been shown to successfully create targeted double-strand breaks in mammalian cells and plant protoplasts. While the versatility of ZFNs and TALEN lies in their ability to be engineered to recognize widely divergent DNA sequences, recent publications show that this versatility can be introduced into other endonucleases. For example, protein engineering has also been applied to homing endonucleases. These “custom” endonucleases derived from I-SceI and its homologs, I-MsoI and I-CreI, have also been shown to target DNA breaks in bacteria, yeast, and mammalian cell lines. More recently Fauser et al. (2012) reported a highly efficient gene targeting system in Arabidopsis that also uses a site-specific endonuclease. The improvement relies on the fact that the enzyme cuts both within the target and the chromosomal trans-genic donor, leading to an excised targeting vector (Fauser F, et al. P Natl Acad Sci USA 2012, 109(19):7535-7540).

Meganucleases

In specific embodiments, the functional editing component is a meganuclease modified to be specific for a target site in the plant genome. In certain embodiments, a genome-editing meganuclease may be used. Meganucleases described herein can be based on the naturally occurring meganuclease I-CreI for use as a scaffold. I-CreI is a homing endonuclease found in the chloroplasts of Chlamydomonas rheinhardti (Thompson et al. 1992, Gene 119, 247-251). This endonuclease is a homodimer that recognizes a pseudo-palindromic 22 bp DNA site in the 23 S rRNA gene and creates a double stranded DNA break that is used from the introduction of an intron. I-CreI is a member of a of group endonucleases carrying a single LAGLIDADG motif. LAGLIDADG enzymes contain one or two copies of the consensus motif. Single-motif enzymes, such as I-CreI are homodimers, whereas double-motif enzymes are monomers with two separate domains. Accordingly, when re-designing meganucleases derived from an I-CreI scaffold to recognize a 22 bp nucleotide sequence of interest, two monomeric units may be designed, each recognizing a part of the 22 bp recognition site, which are needed in concert to induce a double stranded break at the 22 bp recognition site. Concerted action may be achieved by linking the two monomeric units into one single chain meganuclease, or may also be achieved by promoting the formation of heterodimers, as described e.g. in WO2007/047859, incorporated herein by reference.

Accordingly, fusion proteins are disclosed herein in which a peptide linker covalently joins two heterologous LAGLIDADG meganuclease subunits to form a “single-chain heterodimer meganuclease” or “single-chain meganuclease”, in which at least the N-terminal subunit is derived from a mono-LAGLIDADG meganuclease, and in which the subunits function together to preferentially bind to and cleave a non-palindromic DNA recognition site in the genome of a tobacco cell which is a hybrid of the recognition half-sites of the two subunits. In particular, the genetically engineered single-chain meganucleases can be used to recognize non-palindromic DNA sequences that naturally-occurring meganucleases do not recognize in the genome of a tobacco cell. The invention also provides methods that use such meganucleases to produce recombinant nucleic acids and engineered tobacco plants by utilizing the meganucleases to cause recombination of a desired genetic sequence at a limited number of loci within the genome of a tobacco plant, plant part, or plant cell for, inter alia, genetic engineering, protein expression, modulation of nicotine demethylase activity, and in vitro applications in diagnostics and research. See, U.S. Pat. Nos. 9,434,931, 9,340,777, 8,445,251, and 8,338,157, herein incorporated by reference in their entirety.

Thus, in particular embodiments, the methods and compositions disclosed herein utilize recombinant single-chain meganucleases comprising a pair of covalently joined LAGLIDADG subunits derived from one or more mono-LAGLIDADG meganucleases which function together to recognize and cleave a non-palindromic recognition site in the genome of a tobacco cell. In some embodiments, the mono-LAGLIDADG subunit is derived from a wild-type meganuclease selected from I-CreI, I-MsoI and I-CeuI.

CRISPR/Cas

In other embodiments, functional editing components may be part of a RNA-guided endonuclease system, such as the type II CRISPR/Cas system. As used herein an endonuclease system can refer to any endonuclease or combination of endonuclease and other functional editing components capable of introducing a double-strand or single-strand break in the genome of a plant cell. Bacteria and archaea have evolved adaptive immune defenses termed clustered regularly interspaced short palindromic repeats (CRISPR)/CRISPR-associated (Cas) systems that use short RNA to direct degradation of foreign nucleic acids (WO2007/025097). The type II CRISPR/Cas system from bacteria employs a crRNA and tracrRNA to guide the Cas endonuclease to its DNA target. The crRNA (CRISPR RNA) contains the region complementary to one strand of the double strand DNA target and base pairs with the tracrRNA (trans-activating CRISPR RNA) forming a RNA duplex that directs the Cas endonuclease to cleave the DNA target.

In some embodiments, the CRISPR enzyme is a type I or III CRISPR enzyme or the CRISPR enzyme is a type II CRISPR enzyme. This type II CRISPR enzyme may be any Cas enzyme. A preferred Cas enzyme may be identified as Cas9 as this can refer to the general class of enzymes that share homology to the biggest nuclease with multiple nuclease domains from the type II CRISPR system. Most preferably, the Cas9 enzyme is from, or is derived from, spCas9 or saCas9. As used herein a “Cas endonuclease” can be any type II RNA guided DNA endonuclease, such as a Cas9 endonuclease. In one embodiment, the Cas9 nuclease is a SpCas9, SaCas9, NmCas9, or an AnCas9. In particular embodiments, the CRISPR enzyme is a Cpf1 endonuclease which only requires a crRNA to direct cleavage at the target site of the genome. Additionally, the Cpf1 endonuclease creates a staggered double strand break in the genome rather than the blunt end cut created by cleavage by Cas enzymes.

As used herein, the term “guide RNA” relates to any RNA having specificity for a target site in a genome that directs an endonuclease to cleave at the target site. In specific embodiments, a guide RNA is a synthetic fusion of two RNA molecules, a crRNA (CRISPR RNA) comprising a variable targeting domain, and a tracrRNA. In one embodiment, the guide RNA comprises a variable targeting domain of 12 to 30 nucleotide residues and a RNA fragment that can interact with a Cas endonuclease. As used herein, the term “guide polynucleotide”, relates to a polynucleotide sequence that can form a complex with a Cas endonuclease and enables the Cas endonuclease to recognize and optionally cleave a DNA target site. The guide polynucleotide can be a single molecule or a double molecule. The guide polynucleotide sequence can be a RNA sequence, a DNA sequence, or a combination thereof (a RNA-DNA combination sequence). In some embodiments, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA”.

The guide polynucleotide can be a double molecule (also referred to as duplex guide polynucleotide) comprising a first nucleotide sequence domain (referred to as Variable Targeting domain or VT domain) that is complementary to a nucleotide sequence in a target DNA and a second nucleotide sequence domain (referred to as Cas endonuclease recognition domain or CER domain) that interacts with a Cas endonuclease polypeptide. The CER domain of the double molecule guide polynucleotide comprises two separate molecules that are hybridized along a region of complementarity. The two separate molecules can be RNA, DNA, and/or RNA-DNA-combination sequences. In some embodiments, the first molecule of the duplex guide polynucleotide comprising a VT domain linked to a CER domain is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). In some embodiments the second molecule of the duplex guide polynucleotide comprising a CER domain is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides). In one embodiment, the RNA that guides the RNA/Cas9 endonuclease complex, is a duplexed RNA comprising a duplex crRNA-tracrRNA.

The guide polynucleotide can also be a single molecule comprising a first nucleotide sequence domain (referred to as Variable Targeting domain or VT domain) that is complementary to a nucleotide sequence in a target DNA and a second nucleotide domain (referred to as endonuclease recognition domain or CER domain) that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. In some embodiments the single guide polynucleotide comprises a crNucleotide (comprising a VT domain linked to a CER domain) linked to a tracrNucleotide (comprising a CER domain), wherein the linkage is a nucleotide sequence comprising a RNA sequence, a DNA sequence, or a RNA-DNA combination sequence. The single guide polynucleotide being comprised of sequences from the crNucleotide and tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and

DNA nucleotides). In one embodiment, the single guide RNA comprises a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a plant genomic target site, enabling the Cas endonuclease to introduce a double strand break into the genomic target site. One aspect of using a single guide polynucleotide versus a duplex guide polynucleotide is that only one expression cassette needs to be made to express the single guide polynucleotide.

The term “variable targeting domain” or “VT domain” is used interchangeably herein and includes a nucleotide sequence that is complementary to one strand (nucleotide sequence) of a double strand DNA target site. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable target domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” of a guide polynucleotide is used interchangeably herein and includes a nucleotide sequence (such as a second nucleotide sequence domain of a guide polynucleotide), that interacts with a Cas endonuclease polypeptide. The CER domain can be composed of a DNA sequence, a RNA sequence, a modified DNA sequence, a modified RNA sequence, or any combination thereof.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another embodiment, the nucleotide sequence linking the crNucleotide and the tracrNucleotide of a single guide polynucleotide can comprise a tetraloop sequence, such as, but not limiting to a GAAA tetraloop sequence.

Nucleotide sequence modification of the guide polynucleotide, VT domain, and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

In particular embodiments, the guide RNA and Cas endonuclease are capable of forming a complex that enables the Cas endonuclease to introduce a double strand break at a DNA target site. In some embodiments of the disclosure the variable target domain is 12, 13, 14, 15, 16, 17, 18, 19, 20, 21, 22, 23, 24, 25, 26, 27, 28, 29 or 30 nucleotides in length. In one embodiment, the guide RNA comprises a crRNA (or crRNA fragment) and a tracrRNA (or tracrRNA fragment) of the type II CRISPR/Cas system that can form a complex with a type II Cas endonuclease, wherein the guide RNA/Cas endonuclease complex can direct the Cas endonuclease to a plant genomic target site, enabling the Cas endonuclease to introduce a double strand break into the genomic target site.

Compositions for Viral-Based Gene Editing in Plants

Embodiments of the disclosure provide a tobacco mosaic virus (TMV) genome modified to comprise a nucleic acid sequence encoding a meganuclease operably linked to a promoter, The promoter may be a CaMV35S, a T7 RNA polymerase promoter, or a coat protein subgenomic promoter. In certain embodiments, the meganuclease is specific for a target site in a gene encoding a PDS (Phytoene desaturase), nicotine synthase, or a nicotine demethylase. Or other genes may be targeted.

Provided herein are modified TMV (i.e. mTMV) vectors, such as a GENEWARE® pDN15 vector, including at least one functional editing component. Accordingly, in some embodiments, the mTMV vector encodes a functional replicase, a movement protein, and a capsid protein along with a sequence encoding a DNA endonuclease operably linked to a constitutive promoter active in a plant cell. In an embodiment, the functional editing component is a genome-editing endonuclease.

In some embodiments, the mTMV vector comprises at least one of a functional replicase, a movement protein, and a capsid protein along with at least one of a sequence encoding a Cas9 endonuclease operably linked to a constitutive promoter active in a plant cell and a sequence encoding a gRNA operably linked to a constitutive promoter active in a plant cell. In some embodiments the mTMV vector comprises at least one of a functional replicase, a movement protein, and a capsid protein with no functional editing component. The mTMV vector can also comprise at least one of a sequence encoding a Cas9 endonuclease operably linked to a constitutive promoter active in a plant cell and a sequence encoding a gRNA operably linked to a constitutive promoter active in a plant cell without an element of the TMV genome. In particular embodiments a terminator is present at the 3′ of the polynucleotides encoding the Cas9 endonuclease and/or the polynucleotide encoding the gRNA.

In some embodiments, a TMV vector (such as pDN15 or other type of GENEWARE® vector) will provide for expression of a selectable marker gene and targeted endonuclease. In some embodiments, the endonuclease is a meganuclease. In these embodiments, the TMV vector will be preceded by a T7 RNA polymerase promoter operatively linked to the tobamovirus vector genome such that the first transcribed nucleotide promotes capping through in vitro transcription and correct initiation with the virus genome sequence. The vector may be modified with an insertion downstream of the native coat protein subgenomic promoter comprising of a reporter gene, such as the green fluorescent protein, basta resistance (bar) or fusion of the two genes to produce a bi-functional protein. A second insertion may be made downstream of the reporter construct, including a second coat protein subgenomic promoter from a different tobamovirus genome followed by the gene editing endonuclease (including one of the following a meganuclease such as a genome editing endonuclease, TALEN, ZFN, or CRISPR-cas9).

In some embodiments, the vector may designed to lack the coat protein to insure lack of systemic and persistent infection. Additionally and/or alternatively, the vector may terminate with the 3′ non-translated region (NTR) of a tobamovirus due to a ribozyme 3′ of the virus NTR to promoting correct RNA cleavage in transcripts produced in vitro to enhance transcript infectivity. Accordingly, in specific embodiments, viral vectors disclosed herein, encoding an endonuclease as disclosed herein, lack a nucleic acid encoding a coat protein.

In particular embodiments, if a two expression cassette strategy, as described above, is used, the reporter gene and endonuclease sequences can be inserted in reverse order—with endonuclease under the control of the native tobamovirus coat protein subgenomic promoter and the reporter gene under the control of the second tobamovirus coat protein subgenomic promoter. As further modification, the reporter gene (singly or doubly active protein) could be fused to the endonuclease sequence to allow for only a single translated cistron from the native subgenomic promoter. Necessary 3′NTR sequences can be inserted following the endonuclease/reporter gene fusion and upstream of the ribozyme sequence. Guide RNAs can be simultaneously expressed in the TMV-based vectors in three manners: 1) by insertion downstream of selectable marker termination codon; 2) insertion downstream of endonuclease termination codon; 3) or incorporation of a third heterologous subgenomic promoter (e.g. first from tobacco green mottle mosaic virus and second from tomato mosaic virus) and insertion of gRNA sequence downstream of the promoter.

Likewise, provided herein are TMV genomes including at least one functional editing component. Accordingly, in some embodiments, the mTMV genome encodes a functional replicase, a movement protein, and a capsid protein along with a sequence encoding a DNA endonuclease, operably linked to a constitutive promoter active in a plant cell. In some embodiments, the mTMV genome comprises a RNA polymerase, a functional replicase, a movement protein, and a capsid protein along with at least one of a sequence encoding a Cas9 endonuclease operably linked to a constitutive promoter active in a plant cell and a sequence encoding a gRNA operably linked to a constitutive promoter active in a plant cell. In particular embodiments a terminator is present at the 3′ of the polynucleotides encoding the Cas9 endonuclease and/or the polynucleotide encoding the gRNA.

In specific embodiments, TMV vectors, such as pDN15, or other GENEWARE® TMV, or PVX vectors comprise a nucleic acid sequence operably linked to a promoter, such as a coat protein promoter, wherein the nucleic acid sequence encodes a meganuclease such as a genome-editing endonuclease. In some embodiments, multiple vectors or multiple RNA molecules encoding separate meganucleases can be introduced into a plant cell. For example, multiple target sites of a tobacco genome can be modified by introducing nucleic acid molecules, such as RNA molecules, encoding different meganucleases specific for separate target sites.

The compositions and methods disclosed herein utilize a modified TMV genome for delivery of functional editing components to a plant cell. For example, the TMV genome can be modified to deliver a meganuclease to a plant cell or the meganuclease can be expressed in vitro prior to delivery of expressed RNA encoding the meganuclease directly to a plant cell. In some embodiments, the TMV genome can be modified to comprise a detectable marker such as GFP or other known markers. Additionally and/or alternatively, the TMV genome can be modified to comprise a genome-editing endonuclease. In some embodiments, the TMV genome can be modified to comprise a nucleic acid molecule encoding a Cas9 endonuclease and a gRNA or gRNA components for modification of a target site in a plant genome. When the one or more mTMV vectors are delivered to a plant cell and mTMV genome is subsequently expressed, the functional editing components encoded therein can be expressed to facilitate modification of the target site of the plant genome.

Promotors

Any promotor active in a plant cell can be incorporated in a TMV vector for the expression of a functional editing component. In specific alternate embodiments, the promotor is a constitutive promoter, an inducible promoter, a tissue-preferred promoter, a cell type-preferred promoter, or a developmentally-preferred promoter. Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the rubisco promoter, the GRP1-8 promoter and other transcription initiation regions from various plant genes known to those of skill. If low level expression is desired, weak promoter(s) may be used. Weak constitutive promoters include, for example, the core promoter of the Rsyn7 promoter (WO 99/43838 and U.S. Pat. No. 6,072,050), the core 35S CaMV promoter, and the like. Other constitutive promoters include, for example, U.S. Pat. Nos. 5,608,149; 5,608,144; 5,604,121; 5,569,597; 5,466,785; 5,399,680; 5,268,463; and 5,608,142. See also, U.S. Pat. No. 6,177,611, herein incorporated by reference. In some embodiments, the promoter is a CaMV35S, a T7 RNA polymerase promoter, or a coat protein subgenomic promoter.

Examples of inducible promoters are the Adhl promoter which is inducible by hypoxia or cold stress, the Hsp70 promoter which is inducible by heat stress, the PPDK promoter and the pepcarboxylase promoter which are both inducible by light. Also useful are promoters which are chemically inducible, such as the In2-2 promoter which is safener induced (U.S. Pat. No. 5,364,780), the ERE promoter which is estrogen induced, and the Axig1 promoter which is auxin induced and tapetum specific but also active in callus (PCT US01/22169).

Examples of promoters under developmental control include promoters that initiate transcription preferentially in certain tissues, such as leaves, roots, fruit, seeds, or flowers. A “tissue specific” promoter is a promoter that initiates transcription only in certain tissues. Unlike constitutive expression of genes, tissue-specific expression is the result of several interacting levels of gene regulation. As such, promoters from homologous or closely related plant species can be preferable to use to achieve efficient and reliable expression of transgenes in particular tissues. In some embodiments, the expression cassettes comprise a tissue-preferred promoter. A “tissue preferred” promoter is a promoter that initiates transcription mostly, but not necessarily entirely or solely in certain tissues. For example, nucleic acid molecules encoding endolysins or other membrane-disrupting enzymes can be operably linked to leaf-preferred or stem-preferred promoters.

In some embodiments, the expression construct comprises a cell type specific promoter. A “cell type specific” promoter is a promoter that primarily drives expression in certain cell types in one or more organs, for example, vascular cells in roots, leaves, stalk cells, and stem cells. The expression construct can also include cell type preferred promoters. A “cell type preferred” promoter is a promoter that primarily drives expression mostly, but not necessarily entirely or solely in certain cell types in one or more organs, for example, vascular cells in roots, leaves, stalk cells, and stem cells. The expression constructs described herein can also comprise seed-preferred promoters. In some embodiments, the seed-preferred promoters have expression in embryo sac, early embryo, early endosperm, aleurone, and/or basal endosperm transfer cell layer (BETL). Examples of seed-preferred promoters include, but are not limited to, 27 kD gamma zein promoter and waxy promoter, Boronat, A. et al. (1986) Plant Sci. 47:95-102; Reina, M. et al. Nucl. Acids Res. 18(21):6426; and Kloesgen, R. B. et al. (1986) Mol. Gen. Genet. 203:237-244. Promoters that express in the embryo, pericarp, and endosperm are disclosed in U.S. Pat. No. 6,225,529 and PCT publication WO 00/12733.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced expression of an expression construct within a particular plant tissue. Tissue-preferred promoters are known in the art. See, for example, Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters and stem-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant 3:509-18; Orozco et al. (1993) Plant Mol. Biol. 23(6):1129-1138; and Matsuoka et al. (1993) Proc. Natl. Acad. Sci. USA 90(20):9586-9590. In addition, the promoters of cab and rubisco can also be used. See, for example, Simpson et al. (1958) EMBO J 4:2723-2729 and Timko et al. (1988) Nature 318:57-58.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed roIC and roID root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29(4):759-772); and roIB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179. The phaseolin gene (Murai et al. (1983) Science 23:476-482 and Sengopta-Gopalen et al. (1988) PNAS 82:3320-3324).

Other Vector Elements

Additional sequence modifications are known to enhance gene expression in a cellular host. These include elimination of sequences encoding spurious polyadenylation signals, exon-intron splice site signals, transposon-like repeats and other such well-characterized sequences that may be deleterious to gene expression. The G-C content of the heterologous nucleotide sequence may be adjusted to levels average for a given cellular host, as calculated by reference to known genes expressed in the host cell. When possible, the sequence is modified to avoid predicted hairpin secondary mRNA structures.

The mTMV vectors may additionally contain 5′ leader sequences upstream of foreign gene coding regions. Such leader sequences can act to enhance translation. Translation leaders are known in the art and include, without limitation: picornavirus leaders, for example, EMCV leader (Encephalomyocarditis 5′ noncoding region) (Elroy-Stein, et al., (1989) Proc. Nat. Acad. Sci. USA 86:6126-6130); potyvirus leaders, for example, TEV leader (Tobacco Etch Virus) (Allison, et al., (1986) Virology 154:9-20); MDMV leader (Maize Dwarf Mosaic Virus); human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) Molecular Biology of RNA, pages 237-256) and maize chlorotic mottle virus leader (MCMV) (Lommel, et al., (1991) Virology 81:382-385), herein incorporated by reference in their entirety. See, also, Della-Cioppa, et al., (1987) Plant Physiology 84:965-968, herein incorporated by reference in its entirety. Methods known to enhance mRNA stability can also be utilized, for example, introns, such as the maize Ubiquitin intron (Christensen and Quail, (1996) Transgenic Res. 5:213-218; Christensen, et al., (1992) Plant Molecular Biology 18:675-689) or the maize Adhl intron (Kyozuka, et al., (1991) Mol. Gen. Genet. 228:40-48; Kyozuka, et al., (1990) Maydica 35:353-357) and the like, herein incorporated by reference in their entirety.

In preparing the mTMV vectors, the various DNA fragments may be manipulated, so as to provide for the DNA sequences in the proper orientation and, as appropriate, in the proper reading frame. Toward this end, DNA de novo synthesis, DNA adapters or linkers may be employed to join the DNA fragments or other manipulations may be involved to provide for convenient restriction sites, removal of superfluous DNA, removal of restriction sites or the like. For this purpose, in vitro mutagenesis, primer repair, restriction, annealing, resubstitutions, for example, transitions and transversions, may be involved.

Reporter or Selectable Marker Genes

In specific embodiments, the TMV vector can comprise a reporter gene or selectable marker gene. Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification. In certain embodiments, the reporter gene is a GFP or basta resistance gene, or fusion of the two genes to produce a bi-functional protein. For example, the TMV vector can be a pDN15 vector.

In specific embodiments, a GENEWARE® vector, such as a pDN15 vector, can comprise a T7 RNA polymerase promoter operably linked to the TMV genome such that the first transcribed nucleotide promotes capping through in vitro transcription and correct initiation with the viral genome sequence. Further, a pDN15 vector can be modified with a polynucleotide encoding a reporter gene downstream of the native coat protein subgenomic promoter. Downstream of the reporter construct a second TMV coat protein subgenomic promoter can be operably linked to a nucleic acid sequence encoding an endonuclease, such as a meganuclease (e.g., endonuclease A) or Cas9 endonuclease. Finally, a pDN15 vector can have a ribozyme 3′ of the virus NTR to promoter correct RNA cleavage in transcripts produced in vitro. In other embodiments, a gene encoding an endonuclease can be operably linked to the native TMV coat protein subgenomic promoter and a reporter gene can be operably linked to a second TMV coat protein subgenomic promoter. In some embodiments, the reporter gene could be operably linked to the nucleic acid sequence encoding an endonuclease to allow for a single translated cistron from the native subgenomic promoter.

In some embodiments, other TMV vectors could be constructed as described above using genomes of viruses with different or broader host ranges to increase the utility of this transformation system to many dicot and monocot plants. Whereas, different TMV vectors cannot infect the same cells simultaneously, tobamoviruses can super-infect with potyviruses, cucomoviruses, bromoviruses, tobraviruses or potexviruses. A second virus vector composed of a virus from the families described above could be modified to express a second nuclease targeting a second plant gene. The second, non-TMV, vector can be transcribed in vitro and co-infected with the TMV vector described herein. Selection can then proceed for basta resistance expression, and screening of plants for editing of two genes can be screened using genomic sequencing techniques. Conversely, transient basta resistance can be conferred by TMV vectors expressing resistance genes and continuing to replicate transiently in the transfected tissues as tissues are selected for regeneration. In specific embodiments, TMV vectors disclosed herein can be modified to prevent expression of the coat protein.

Methods for Viral Based Gene Editing

Methods for modifying a plant genomic target site are disclosed herein. For example, in certain embodiments disclosed is a method for modifying a target site in the genome of a tobacco plant cell, the method comprising: introducing a nucleic acid encoding a functional editing component into the tobacco plant cell, wherein the functional editing component introduces a modification at the target site in the genome of the tobacco plant cell. In certain embodiments, the functional editing component is an endonuclease that cleaves DNA. The endonuclease may be one of a meganuclease and/or a guide RNA and/or Cas9 endonuclease. In some embodiments, the nucleic acid comprises an RNA expression vector. For example, in some embodiments the vector is a tobacco mosaic virus (TMV) vector.

The functional editing component may be operably linked to a promoter. In certain embodiments, the promoter is one of a CaMV35S, a T7 RNA polymerase promoter, or a coat protein subgenomic promoter. As discussed in detail herein, the nucleic acid may be synthesized in the plant cell or may be synthesized in vitro prior to introducing the nucleic acid nucleic acid encoding a functional editing component into the plant cell.

Thus in some embodiments, a method for modifying a target site in the genome of a plant cell comprises introducing at least one TMV vector modified to express a functional editing component. In an embodiment, the functional editing component is a genome-editing endonuclease. In an alternate embodiment, the method for modifying a target site in the genome of a plant cell comprises introducing at least one TMV vector modified to express a guide RNA into a plant cell having a Cas endonuclease as the functional editing components, wherein said guide RNA and Cas endonuclease are capable of forming a complex that enables the Cas endonuclease to introduce a double strand break at the target site.

Once a double-strand break is induced in the DNA, the cell's DNA repair mechanism is activated to repair the break. Error-prone DNA repair mechanisms can produce mutations at double-strand break sites. The most common repair mechanism to bring the broken ends together is the nonhomologous end-joining (NHEJ) pathway (Bleuyard et al., (2006) DNA Repair 5:1-12). The structural integrity of chromosomes is typically preserved by the repair, but deletions, insertions, or other rearrangements are possible and common (Siebert and Puchta, (2002) Plant Cell 14:1121-31; Pacher et al., (2007) Genetics 175:21-9). A double-strand break can also be repaired by homologous recombination (HR) between homologous DNA sequences. Once the sequence around the double-strand break is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al., (2004) Plant Cell 16:342-52). Ectopic and/or epigenic DNA sequences may also serve as a DNA repair template for homologous recombination (Puchta, (1999) Genetics 152:1173-81).

Homology-directed repair (HDR) is a mechanism in cells to repair double-stranded and single stranded DNA breaks. Homology-directed repair includes homologous recombination (HR) and single-strand annealing (SSA) (Lieber. 2010 Annu. Rev. Biochem. 79:181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks (Davis and Maizels. PNAS (0027-8424), 111 (10), p. E924-E932.

Alteration of the genome of a plant cell, for example, through homologous recombination (HR), is a powerful tool for genetic engineering. Despite the low frequency of homologous recombination in higher plants, there are examples of successful homologous recombination of plant endogenous genes. The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the plant genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination.

“Donor DNA” can be used to repair a double stranded break or insert a polynucleotide of interest at a double stranded break site. Thus, donor DNA can be heterologous to the target site and can be provided on a mTMV vector or expressed from a mTMV genome in a plant cell. The term “heterologous” according to the present invention when used in reference to a sequence is intended to mean a sequence that originates from a species other than the species in which it is to be expressed, or, if from the same species as the species in which it is to be expressed, is substantially modified from its native form in composition and/or genomic locus by deliberate human intervention.

The region of homology on the donor DNA can have homology to any sequence flanking the target site. While in some embodiments the regions of homology share significant sequence homology to the genomic sequence immediately flanking the target site, it is recognized that the regions of homology can be designed to have sufficient homology to regions that may be further 5′ or 3′ to the target site. In still other embodiments, the regions of homology can also have homology with a fragment of the target site along with downstream genomic regions. In one embodiment, a first region of homology further comprises a first fragment of the target site and a second region of homology comprises a second fragment of the target site, wherein the first and second fragments are dissimilar.

In one embodiment, a guide polynucleotide/Cas endonuclease system or the endonuclease A is used for introducing one or more polynucleotides of interest or one or more traits of interest into one or more target sites by providing one or more donor DNAs to a plant cell. A fertile plant can be produced from that plant cell that comprises an alteration at said one or more target sites, wherein the alteration is selected from the group consisting of (i) replacement of at least one nucleotide, (ii) a deletion of at least one nucleotide, (iii) an insertion of at least one nucleotide, and (iv) any combination of (i)-(iii). In particular embodiments, a target site can be located within a polynucleotide encoding a protein or trait of interest such that cleavage by the RNA-guided endonuclease (e.g., Cas9 or endonuclease A) at the target site can prevent expression of the protein or trait of interest. In some embodiments, plants comprising these altered target sites can be crossed with plants comprising at least one gene or trait of interest in the same complex trait locus, thereby further stacking traits in said complex trait locus (see also, US-2013-0263324-A1).

In one embodiment provided herein, the method for editing a target site in a plant genome comprises contacting a plant cell with a mTMV vector comprising functional editing components, such as a nucleic acid molecule encoding a meganuclease, or at least two mTMV vectors together comprising all functional editing components necessary for genome modification. In an embodiment, a GENEWARE® vector, such as a modified RJRTARL002 is used.

Following assembly of the mTMV and expression of the functional editing components, such as a meganuclease, a double-strand break can be introduced in the target site by the encoded endonuclease. In some embodiments, the nucleic acid molecule, such as a RNA molecule, encoding a meganuclease can be synthesized in vitro from a mTMV vector and delivered directly to the plant cell wherein a double-strand break can be introduced in the target site by the encoded meganuclease. In specific embodiments, the double-strand break can be repaired by NHEJ, thereby inactivating any coding sequence comprising the target site. In particular embodiments, a polynucleotide of interest flanked by a first and second region of homology can be inserted into the plant genome at the target site by homologous recombination. Specifically, the first and second regions of homology of the donor DNA can undergo homologous recombination with their corresponding genomic regions of homology resulting in exchange of DNA between the donor and the genome. As such, the provided methods result in the integration of the polynucleotide of interest of the donor DNA into the double-strand break in the target site in the plant genome, thereby altering the original target site and producing an edited genomic target site.

A mTMV vector, such as but not limited to a modified GENEWARE® vector such as the vectors disclosed here (e.g., RJRTARL002 or a modified RJRTARL002 modified to include a DNA fragment that encodes a genome-editing endonuclease) or multiple mTMV vectors comprising a functional editing component may be introduced by any means known in the art for introduction of TMV into a plant cell having a target site. In order to convert TMV into an expression vector, an additional subgenomic promoter can be inserted into the viral genome to drive the expression of an inserted foreign gene, such as a functional editing component. Accordingly, a “TMV vector” or “mTMV vector” is a TMV genome modified to express at least one functional editing component. For example, a mTMV vector can comprise a polynucleotide encoding a meganuclease, a RNA-guided DNA endonuclease, a polynucleotide encoding a complete gRNA specific for a target site, a polynucleotide encoding a crRNA, a polynucleotide encoding a tracrRNA, or any combination thereof. In particular embodiments, the functional editing component on the mTMV vector can be operably linked to a promoter active in a plant cell. In some embodiments, the mTMV vector is located on a Ti plasmid used for Agrobacterium infection. For example, the Ti plasmid can comprise the mTMV vector, an origin of replication, and a virulence region, among other known regions that participate in the transfer of genetic material from Agrobacterium into plants (White et al., Plant Biotechnology, Kung and Arntzen eds. Butterworth Pub., Boston, Mass., 1989).

Infecting and Culturing Plants

The methods described herein can include infecting tobacco plants having a target site of interest with mTMV vectors using one or more of agroinfiltration or agroinfection procedures. Also, the methods can include introducing the mTMV vectors described herein by performing pressure infiltration of plant tissues, hand inoculation of a surface of a leaf (e.g., rubbing), a mechanical inoculation of a plant bed, a high pressure spray of a leaf, or a vacuum infiltration. In specific embodiments, a nucleic acid molecule encoding an endonuclease is delivered directly to the plant cell by mechanical transmission means. For example, a RNA molecule encoding a meganuclease synthesized from a GENEWARE® vector in vitro, can be delivered to the plant cell by rubbing, high pressure spray, gene gun, or similar technologies. The GENEWARE® vector can be modified to remove cryptic splice-sites and have introns added to promote release from nucleus. Such a TMV vector encoding a meganuclease can also be delivered directly to a plant cell wherein the meganuclease is expressed from the vector in the plant cell. See, Pogue et al., 2010. Gleba & Giritch in Recent Advances in Plant Virology. eds., Caranta, Tepfer, & Lopez-Moya. Norfolk, Caister Academic Press 2011:387-412, herein incorporated by reference.

In some embodiments, mTMV described herein is infected into a plant via Agrobacterium transformation through leaf infiltration. The functional editing components of an endonuclease system (e.g., CRISPR/Cas or meganuclease) can be provided on a single mTMV vector or different functional editing components can be provided on separate viral vectors such as TVCV and PVX so that, upon infection, each functional editing component is expressed to result in an active endonuclease system capable of editing a target site of the plant genome. Following leaf infiltration the mTMV vector alone or mTMV and PVX vectors can express the mTMV genome elements to produce an assembled mTMV capable of replicating within the leaf tissue and spreading to adjacent leaf tissue. During replication and spreading of the mTMV, the encoded endonuclease system can introduce modifications at the target site of the plant genome. In some embodiments, the plant infected with mTMV as described herein can be cultured until the plant flowers. After flowering, the plant can be cultured until seeds are produced comprising an edit at the target site of the genome of the seed induced by expression of the endonuclease and other functional editing components. Seeds comprising an edit at the target site of the genome can be isolated and subsequently cultured to produce a plant having the genome edit at the target site in all, or substantially all, of the plant cells with no remaining mTMV vector or TMV.

In specific embodiments, parts of a plant (e.g., the leaf, meristem, shoot, and/or flower of the plant) having the RNA molecule encoding a DNA endonuclease can be harvested and cultured on selective media as provided by RNA transcripts of the TMV vector or agroinfiltration of a DNA-based tobamovirus vector. For example, the parts of the plant surrounding the introduction site of the RNA molecule can be removed from the plant and cultured on selective media. In some embodiments, the part of the plant is a leaf part having the RNA molecule or the TMV genome expressing a meganuclease is a part of the leaf. In some embodiments, the selective media contains basta. Resistance is transient due to the non-DNA-based nature of the TMV vector and its inability to systemically infect plant tissue. It provides basta resistance protein sufficiently for plant tissue selection and then is no longer propagated in the growing and maturing plantlet.

In certain embodiments, mTMV can be harvested from the leaf tissues following leaf infiltration of a single mTMV vector expressing a functional endonuclease system or infiltration of separate mTMV vectors collectively expressing a functional endonuclease system. In some embodiments, mTMV can be harvested from plant leaves or any other plant part at least 2, 3, 4, 5, 6, 7, 8, 9, 10, 12, 14, 16, 18, 21, 28, 35, or 42 days post infection. mTMV can be harvested from plant tissue, (e.g. leaf tissue) by any method known in the art for harvesting TMV from plant tissue. In specific embodiments, leaf tissue infected with mTMV can be ground in the presence of acetate buffer, heated to about 42° C. and centrifuged to clarify the extract. mTMV can be harvested as a complete and functional virus from the site of infection, even if elements of the mTMV genome were delivered on separate mTMV vectors. In some embodiments mTMV can be harvested from the meristem, shoot, and/or flower of the plant, such as the tobacco plant.

In particular embodiments, plant parts are selected for propagation based on accumulation of mTMV in the particular part. For example, plants parts wherein accumulation of viral vectors encoding a functional endonuclease occurs can be removed or harvested from the plant for further cultivation in tissue culture. In specific embodiments, the meristem, shoot, and/or flower accumulate viral vector encoding a functional endonuclease and are harvested for propagation in tissue culture. In some embodiments, GFP expression from the viral vector can be used to help identify plant parts having accumulation of viral vector expressing a functional endonuclease. The plant parts propagated in tissue culture can be grown into plants according to methods known in the art.

Following harvesting, the harvested mTMV can be used to infect a second plant. In specific embodiments, mTMV harvested from an infected plant is used to infect plant seedlings of a second plant. The infected seedlings can then be cultured during which time the mTMV can express a complete endonuclease system to edit a target site of the plant genome. For example, the infected seedlings can be cultured until the plant flowers and produces seeds comprising an edit at the target site of the genome of the seed induced by expression of the Cas endonuclease and other functional editing components. Seeds comprising an edit at the target site of the genome can be isolated and subsequently cultured to produce a plant having the genome edit at the target site in all, or substantially all, of the plant cells.

In some embodiments, tobacco seeds having a genome comprising an edit at the target site of the genome can undergo embryo rescue or other virus removal processes. As used herein, embryo rescue is the process plant breeders use to attempt to germinate embryos that may be weak, immature, or would otherwise not develop into a mature viable seed on the parent plant. For example, one form of embryo rescue is ovule culture, which involves aseptically removing the ovule from the seed and placing the ovule onto artificial media to enable the embryo to germinate and grow into a plant. Thus, following embryo rescue or other virus removal process, a plant having an edit at the genome target site is produced, without any remaining functional mTMV or TMV vector.

Target Sites Target sites of interest in the genome of tobacco plant cells can be located in a polynucleotide encoding a trait of interest or encoding a pathway that participates in a trait of interest. The length of the target site can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence.

In some embodiments, a target site can be located in polynucleotide of interest, such as herbicide-resistance coding sequences, insecticidal coding sequences, nematicidal coding sequences, antimicrobial coding sequences, antifungal coding sequences, antiviral coding sequences, abiotic and biotic stress tolerance coding sequences, or sequences modifying plant traits such as yield, grain quality, nutrient content, starch quality and quantity, nitrogen fixation and/or utilization, fatty acids, and oil content and/or composition. More specific polynucleotides of interest include, but are not limited to, genes that improve crop yield, polypeptides that improve desirability of crops, genes encoding proteins conferring resistance to abiotic stress, such as drought, nitrogen, temperature, salinity, toxic metals or trace elements, or those conferring resistance to toxins such as pesticides and herbicides, or to biotic stress, such as attacks by fungi, viruses, bacteria, insects, and nematodes, and development of diseases associated with these organisms. General categories of polynucleotides of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, fertility or sterility, grain characteristics, and commercial products. Genes of interest include, generally, those involved in oil, starch, carbohydrate, or nutrient metabolism as well as those affecting kernel size, sucrose loading, and the like that can be stacked or used in combination with other traits, such as but not limited to herbicide resistance, described herein.

In specific embodiments, the target site can be located in a nicotine demethylase that are involved in the metabolic conversion of nicotine to nornicotine in the roots of tobacco plants. Reducing the activity of a nicotine demethylase by modifying a target site within the gene could reduce the level of Tobacco Specific Nitrosamines (TSNAs) in tobacco products produced by the modified plant containing reduced nicotine demethylase activity. For example, the nicotine demethylase could be CYP82E2, CYP82E21, CYP82E10, CYP82E3, CYP82E4, or CYP82E5. See, for example, U.S. Patent Application Publication 20150315603. The target site can also be located in genes encoding phytoene desaturase (PDS), or any gene of the nicotine synthesis pathway, such as nicotine synthase or nicotine demethylase. In particular embodiments, the target site can be located in a gene involved with alkaloid biosynthesis. For example, genes encoding proteins that participate in the alkaloid biosynthesis pathway that could contain a target site for the nucleases disclosed herein include, but are not limited to: quinolinate phosphoribosyltransferase (QPT), isoflavone reductase (A622), berberine bridge enzyme (BBL), nicotine N-demethylase (NND), N-methylputrescine oxidase (MPO), putrescine methyltransferase (PMT), ornithine decarboxylase (ODC), and arginine decarboxylase (ADC). See, for example, Dewey and Xie, Phytochemistry 94(2013): 10-27, herein incorporated by reference.

In some embodiments, polynucleotides of interest can be inserted at the target site following cleavage by the meganuclease and/or RNA-guided endonuclease disclosed herein. Polynucleotides of interest can be provided on a mTMV vector or on a separate expression vector provided to the plant cell. In some embodiments, the polynucleotide of interest is flanked to a first and second homology arm in order to provide an opportunity for homologous recombination. In particular embodiments, the first homology arm is homologous to a DNA region at the 5′ end of the target site and the second homology arm is homologous to a region at the 3′ end of the target site. In other embodiments, the first homology arm is homologous to a DNA region at the 3′ end of the target site and the second homology arm is homologous to a region at the 5′ end of the target site.

As used herein, a homology arm and a target site “correspond” or are “corresponding” to one another when the two regions share a sufficient level of sequence identity to one another to act as substrates for a homologous recombination reaction. By “homology” is meant DNA sequences that are either identical or share sequence identity to a corresponding sequence. The sequence identity between a given target site and the corresponding homology arm found on the targeting vector can be any degree of sequence identity that allows for homologous recombination to occur. For example, the amount of sequence identity shared by the homology arm (or a fragment thereof) and the target site (or a fragment thereof) can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination. Moreover, a corresponding region of homology between the homology arm and the corresponding target site can be of any length that is sufficient to promote homologous recombination at the cleaved recognition site. For example, a given homology arm and/or corresponding target site can comprise corresponding regions of homology that are from about 400 bp to about 500 bp, from about 500 bp to about 600 bp, from about 600 bp to about 700 bp, from about 700 bp to about 800 bp, from about 800 bp to about 900 bp, or from about 900 bp to about 1000 bp such that the homology arm has sufficient homology to undergo homologous recombination with the corresponding target sites within the genome of the cell.

Expression of Proteins of Interest

Polynucleotide sequences of interest may encode proteins involved in providing disease or pest resistance. By “disease resistance” or “pest resistance” is intended that the plants avoid the harmful symptoms that are the outcome of the plant-pathogen interactions. Pest resistance genes may encode resistance to pests that have great yield drag such as rootworm, cutworm, European Corn Borer, and the like. Disease resistance and insect resistance genes such as lysozymes or cecropins for antibacterial protection, or proteins such as defensins, glucanases or chitinases for antifungal protection, or Bacillus thuringiensis endotoxins, protease inhibitors, collagenases, lectins, or glycosidases for controlling nematodes or insects are all examples of useful gene products. Genes encoding disease resistance traits include detoxification genes, such as against fumonisin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like. Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); and the like.

An “herbicide resistance protein” or a protein resulting from expression of an “herbicide resistance-encoding nucleic acid molecule” includes proteins that confer upon a cell the ability to tolerate a higher concentration of an herbicide than cells that do not express the protein, or to tolerate a certain concentration of an herbicide for a longer period of time than cells that do not express the protein. Herbicide resistance traits may be introduced into plants by genes coding for resistance to herbicides that act to inhibit the action of acetolactate synthase (ALS), in particular the sulfonylurea-type herbicides, genes coding for resistance to herbicides that act to inhibit the action of glutamine synthase, such as phosphinothricin or basta (e.g., the bar gene), glyphosate (e.g., the EPSP synthase gene and the GAT gene), HPPD inhibitors (e.g, the HPPD gene) or other such genes known in the art. See, for example, U.S. Pat. Nos. 7,626,077, 5,310,667, 5,866,775, 6,225,114, 6,248,876, 7,169,970, 6,867,293, and U.S. Provisional Application No. 61/401,456, each of which is herein incorporated by reference. The bar gene encodes resistance to the herbicide basta, the nptll gene encodes resistance to the antibiotics kanamycin and geneticin, and the ALS-gene mutants encode resistance to the herbicide chlorsulfuron.

Furthermore, it is recognized that the polynucleotide of interest may also comprise antisense sequences complementary to at least a portion of the messenger RNA (mRNA) for a targeted gene sequence of interest. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

In some embodiments, the polynucleotide of interest may also be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity. See, U.S. Pat. Nos. 5,283,184 and 5,034,323; herein incorporated by reference.

The polynucleotide of interest can also be a phenotypic marker. A phenotypic marker is screenable or a selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like. In specific embodiments a gene encoding GFP can be inserted at the target site.

Plants

The methods and compositions disclosed herein can be used to edit a target site of the genome of any plant of interest. In specific embodiments, the plants used in the methods and compositions disclosed herein are tobacco plants. For example, in some embodiments, at least one RNA molecule disclosed herein is introduced into a tobacco plant. Any tobacco species can be modified according to the methods disclosed herein. “Tobacco” or “tobacco plant” refers to any species in the Nicotiana genus that produces nicotinic alkaloids. In certain embodiments, tobaccos that can be employed include flue-cured or Virginia (e.g., K326), burley (i.e., light air cured), sun-cured (e.g., Indian Kurnool and Oriental tobaccos, including Katerini, Prelip, Komotini, Xanthi and Yambol tobaccos), Maryland, dark, dark-fired, dark air cured (e.g., Pasado, Cubano, Jatim and Bezuki tobaccos), light air cured (e.g., North Wisconsin and Galpao tobaccos), Indian air cured, Red Russian and Rustica tobaccos, as well as various other rare or specialty tobaccos and various blends of any of the foregoing tobaccos. Descriptions of various types of tobaccos, growing practices and harvesting practices are set forth in Tobacco Production, Chemistry and Technology, Davis et al. (Eds.) (1999), which is incorporated herein by reference. Various representative other types of plants from the Nicotiana genus are set forth in Goodspeed, The Genus Nicotiana, (Chonica Botanica) (1954); U.S. Pat. No. 4,660,577 to Sensabaugh, Jr. et al.; U.S. Pat. No. 5,387,416 to White et al. and U.S. Pat. No. 7,025,066 to Lawson et al.; US Patent Appl. Pub. Nos. 2006/0037623 to Lawrence, Jr. and 2008/0245377 to Marshall et al.; each of which is incorporated herein by reference. Exemplary Nicotiana species include N. tabacum, N. rustica, N. alata, N. arentsii, N. excelsior, N. forgetiana, N. glauca, N. glutinosa, N. gossei, N. kawakamii, N. knightiana, N. langsdorffi, N. otophora, N. setchelli, N. sylvestris, N. tomentosa, N. tomentosiformis, N. undulata, N. x sanderae, N. africana, N. amplexicaulis, N. benavidesii, N. bonariensis, N. debneyi, N. longiflora, N. maritina, N. megalosiphon, N. occidentalis, N. paniculata, N. plumbaginifolia, N. raimondii, N. rosulata, N. simulans, N. stocktonii, N. suaveolens, N. umbratica, N. velutina, N. wigandioides, N. acaulis, N. acuminata, N. attenuata, N. benthamiana, N. cavicola, N. clevelandii, N. cordifolia, N. corymbosa, N. fragrans, N. goodspeedii, N. linearis, N. miersii, N. nudicaulis, N. obtusifolia, N. occidentalis subsp. Hersperis, N. pauciflora, N. petunioides, N. quadrivalvis, N. repanda, N. rotundifolia, N. solanifolia, and N. spegazzinii. As used herein, non-burley tobacco is any variety that is not a burley variety. Accordingly, one of skill in the art would understand that the methods and compositions disclosed herein can be used to modify the genome of any member of the Solanaceae family.

Nicotiana species can be derived using genetic-modification or crossbreeding techniques (e.g., tobacco plants can be genetically engineered or crossbred to increase or decrease production of components, characteristics or attributes). See, for example, the types of genetic modifications of plants set forth in U.S. Pat. No. 5,539,093 to Fitzmaurice et al.; U.S. Pat. No. 5,668,295 to Wahab et al.; U.S. Pat. No. 5,705,624 to Fitzmaurice et al.; U.S. Pat. No. 5,844,119 to Weigl; U.S. Pat. No. 6,730,832 to Dominguez et al.; U.S. Pat. No. 7,173,170 to Liu et al.; U.S. Pat. No. 7,208,659 to Colliver et al. and U.S. Pat. No. 7,230,160 to Benning et al.; US Patent Appl. Pub. No. 2006/0236434 to Conkling et al.; and PCT WO 2008/103935 to Nielsen et al. See, also, the types of tobaccos that are set forth in U.S. Pat. No. 4,660,577 to Sensabaugh, Jr. et al.; U.S. Pat. No. 5,387,416 to White et al.; and U.S. Pat. No. 6,730,832 to Dominguez et al., each of which is incorporated herein by reference. The genetically modified plants of the genus Nicotiana as described herein are suitable for conventional growing and harvesting techniques, such as cultivation in manure rich soil or without manure, bagging the flowers or no bagging, or topping or no topping. The harvested leaves and stems may be used in any traditional tobacco product including, but not limited to, pipe, cigar and cigarette tobacco, and chewing tobacco in any form including leaf tobacco, shredded tobacco, or cut tobacco.

A “control” or “control plant” or “control plant cell” provides a reference point for measuring changes in phenotype of the subject plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which does not express a functional editing component described herein); (c) a plant or plant cell which is a non-transformed segregant among progeny of a subject plant or plant cell; or (d) the subject plant or plant cell itself, under conditions in which heterologous nucleic acids encoding an functional editing component not expressed. Similarly, a “control tobacco product” can refer to a tobacco product produced with tobacco plants or plant parts with no edit at a given target site.

Tobacco plant cells that have been edited at a genomic target site, as disclosed herein can be grown into whole plants. The regeneration, development, and cultivation of plants from single plant protoplast transformants or from various transformed explants is well known in the art. See, for example, McCormick et al. (1986) Plant Cell Reports 5:81-84; Weissbach and Weissbach, In: Methods for Plant Molecular Biology, (Eds.), Academic Press, Inc. San Diego, Calif., (1988). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. Two or more generations may be grown to ensure that expression of the desired phenotypic characteristic is stably maintained and inherited and then seeds harvested to ensure expression of the desired phenotypic characteristic has been achieved. In this manner, the compositions presented herein provide transformed seed (also referred to as “transgenic seed”) having a polynucleotide provided herein, for example, a recombinant miRNA expression construct, stably incorporated into their genome.

In specific embodiments, at least one mTMV vector encoding a functional editing component can be introduced by Agrobacterium transformation through leaf infiltration. The resulting plant can then be allowed to flower and genome-edited seeds can be harvested and grown into a clean genome-edited plant without mTMV remaining in the plant cell. In specific embodiments, genome-edited seeds can undergo embryo rescue or other virus removal process. Subsequently, a Nicotiana plant or plant part grown from genome-edited seeds can be selected using methods known to those of skill in the art such as, but not limited to, Southern blot analysis, DNA sequencing, PCR analysis, or phenotypic analysis. A plant or plant part edited by the foregoing embodiments is grown under plant forming conditions. Plant forming conditions are well known in the art.

Tobacco Products

A plant grown from genome-edited seeds can be subsequently harvested and used for the production of tobacco products. For example, after harvesting, tobacco plants and/or leaves can be fermented. Exemplary fermentation processes for tobacco are provided in U.S. Pat. No. 2,927,188 to Brenik et al.; U.S. Pat. No. 4,660,577 to Sensabaugh et al.; U.S. Pat. No. 4,528,993 to Sensabaugh et al.; and U.S. Pat. No. 5,327,149 to Roth et al., which are incorporated herein by reference. Fermentation is understood to be enhanced by the presence of, e.g., Lactobacillus; consequently, modification of the amount of Lactobacillus bacteria associated with a given sample (e.g., by means of a lactic acid bacteria treatment solution as disclosed above) can, in some embodiments, impact the fermentation of that sample. Where that treated tobacco is later subjected to fermentation, the fermentation can, in some embodiments, be enhanced by the presence of a greater number of Lactobacillus bacteria. In some embodiments, the Lactobacillus bacterium expresses an endolysin or other membrane-disrupting enzyme. By “enhanced” is meant that the fermentation process proceeds, for example, more quickly, and/or more uniformly.

When the fermentation is completed to the desired extent, the fermented tobacco material is typically treated with heat. This heat treatment can, in some embodiments, be sufficient to stop the fermentation and heat kill any active, vegetative microbes. This post-fermentation heat treatment can be achieved, for example, in a manner similar to that described above with respect to heat treatment prior to fermentation. In some embodiments, various components can then be added to the heat treated fermented tobacco material. For example, preservatives, casings, moisture, and salinity can be adjusted through addition of the appropriate components to the heat treated fermented tobacco material (e.g., by adding such components directly to the fermentation vessel). Alternatively, in some embodiments certain components can be added prior to fermentation when it is advantageous to adjust the pool of reagents prior to fermentation. In certain embodiments, following the method disclosed above, the heat treated tobacco material is dried (e.g., to a moisture level of between about 15% and about 20%, e.g., about 18% moisture) for storage and shipping. Such heat treated tobacco material can be subsequently processed, e.g., by adjusting the final salinity, preservative, casing and moisture content.

After treatment, the treated tobacco material can be used in a green form (e.g., the plant or portion thereof can be used without being subjected to any curing process). For example, the plant or portion thereof can be used without being subjected to significant storage, handling or processing conditions. In certain situations, it is advantageous that the plant or portion thereof be used virtually immediately after harvest. Alternatively, for example, a plant or portion thereof in green form can be refrigerated or frozen for later use, freeze dried, subjected to irradiation, yellowed, dried, cured (e.g., using air drying techniques or techniques that employ application of heat), heated or cooked (e.g., roasted, fried or boiled), or otherwise subjected to storage or treatment for later use. It is understood that the benefits, e.g., reduced TSNA formation, enhanced fermentation, and the like, are realized after curing; therefore, the treated materials described herein are advantageously cured prior to use, e.g., in a tobacco product. Tobacco compositions intended to be used in a combustible or smokeless form may incorporate a single type of tobacco (e.g., in a so-called “straight grade” form). For example, the tobacco within a tobacco composition may be composed solely of flue-cured tobacco (e.g., all of the tobacco may be composed, or derived from, either flue-cured tobacco lamina or a mixture of flue-cured tobacco lamina and flue-cured tobacco stem. The tobacco within a tobacco composition also may have a so-called “blended” form. For example, the tobacco within a tobacco composition of the present invention may include a mixture of parts or pieces of flue-cured, burley (e.g., Malawi burley tobacco) and Oriental tobaccos (e.g., as tobacco composed of, or derived from, tobacco lamina, or a mixture of tobacco lamina and tobacco stem). For example, a representative blend may incorporate about 30 to about 70 parts burley tobacco (e.g., lamina, or lamina and stem), and about 30 to about 70 parts flue cured tobacco (e.g., stem, lamina, or lamina and stem) on a dry weight basis. Other exemplary tobacco blends incorporate about 75 parts flue-cured tobacco, about 15 parts burley tobacco, and about 10 parts Oriental tobacco; or about 65 parts flue-cured tobacco, about 25 parts burley tobacco, and about 10 parts Oriental tobacco; or about 65 parts flue-cured tobacco, about 10 parts burley tobacco, and about 25 parts Oriental tobacco; on a dry weight basis. Other exemplary tobacco blends incorporate about 20 to about 30 parts Oriental tobacco and about 70 to about 80 parts flue-cured tobacco.

The tobacco materials provided according to the present disclosure can be further processed and used in ways generally known in the art. See, for example, U.S. Patent Appl. Publ. Nos. 2012/0272976 to Byrd et al. and 2014/0299136 to Moldoveanu et al., which are incorporated herein by reference. In various embodiments, the tobacco can be employed in smoking articles, smokeless tobacco products, and electronic smoking articles.

Thus, in certain embodiments, the modified tobacco plants disclosed herein comprising an edit at a target site when compared to a control tobacco plant can be harvested and processed into a tobacco product. As used herein a tobacco product includes leaf tobacco, shredded tobacco, cut tobacco, ground tobacco, powder tobacco, tobacco extract, nicotine extract, smokeless tobacco, moist or dry snuff, kretek, pipe tobacco, cigar tobacco, cigarillo tobacco, cigarette tobacco, chewing tobacco, bidis, bits, cigarette, cigarillo, a non-ventilated recess filter cigarette, a vented recess filter cigarette, a cigar, and tobacco-containing gum, lozenges, patches, electronic cigarettes, or any combination thereof. In certain embodiments, tobacco products provided herein comprise a decreased nicotine content compared to corresponding tobacco products produced by tobacco plants or plant parts not modified using the mTMV vectors disclosed herein.

The following examples are provided to illustrate further aspects associated with the present disclosure, but should not be construed as limiting the scope thereof. Unless otherwise noted, all parts and percentages are by dry weight.

### EXAMPLES

### Example 1—Viral RNA Mediated Transformation System

Vector construction: A GENEWARE® TMV vector (such as pDN15) is preceded by a T7 RNA polymerase promoter operatively linked to the tobamovirus vector genome such that the first transcribed nucleotide promotes capping through in vitro transcription and correct initiation with the virus genome sequence. The vector (such as pDN15) is modified with an insertion downstream of the native coat protein subgenomic promoter comprising of a reporter gene, such as the green fluorescent protein (GFP), basta resistance (bar), or fusion of the two genes to produce a bi-functional protein. A second insertion is made downstream of the reporter construct, including a second coat protein subgenomic promoter from a different tobamovirus genome followed by the gene editing endonuclease (including one of the following TALEN, ZFN, CRISPR-cas9, or a meganuclease). The vector (such as pDN15) lacks the coat protein to insure lack of systemic and persistent infection and terminates with the 3′ non-translated region (NTR) of a tobamovirus. Finally, the vector (such as pDN15) encodes a ribozyme 3′ of the virus NTR to promote correct RNA cleavage in transcripts produced in vitro to enhance transcript infectivity. If a two expression cassette strategy is used, the reporter gene and endonuclease sequences are inserted in reverse order. The endonuclease is under the control of the native tobamovirus coat protein subgenomic promoter and the reporter gene under the control of the second tobamovirus coat protein subgenomic promoter. As further modification, the reporter gene (singly or doubly active protein) can be fused to the endonuclease sequence to allow for only a single translated cistron from the native subgenomic promoter. Necessary 3′NTR sequences are inserted following the endonuclease/reporter gene fusion and upstream of the ribozyme sequence.

### Example 2—Gene Editing Procedure

Infectious vector RNA is synthesized in vitro with T7 promoter. Synthesized RNA is delivered into plant leaf cells by direct mechanical transmission using rubbing, high pressure spray, gene gun or similar technologies. The synthesized RNA serves as the shuttle containing the endonuclease RNA sequence. Virus transcripts are translated in infected plant cells producing replication proteins to multiple genomic RNAs and produce subgenomic RNA sequences. The RNA undergoes replication to produce subgenomic RNAs including reporter gene and endonuclease encoding RNAs. These are translated to provide reporter for infection (visual or through herbicide selection) and the endonuclease protein, which edits the plant genome in a site specific manner.

Leaf materials can be used for selection of infection sites in situ or through cell culture. For in situ selection, basta is sprayed on leaf surface, infected leaf tissues where virus vector RNA containing the basta resistance gene is replicating is indicated by living green leaf material after 24-48 hrs. Green leaf tissue is excised and cultured on selection regeneration media containing basta. Leaf tissues are visualized using long UV light to identify areas with GFP expression. These areas are then excised and applied on selection regeneration media containing basta. Using either method, rooted shoots are produced and transferred to growth media or soil, then a number of seeds are produced without any viral or genome editing protein sequences.

### Example 3—Generation of a GENEWARE® Vectors and Expression of a GFP Reporter Protein in Transformed Plant Cells

A. Generation of a GENEWARE® Recombinant Vector with GFP

In these experiments, the GENEWARE® vector derived from Tobacco Mosaic Virus (TMV) was used. Using the GENEWARE® vector, a gene encoding foreign protein can be inserted in place of the virus coat protein (CP), so it will be driven by the endogenous virus CP promoter and overexpressed in plant cells (Pogue et al., 2010).

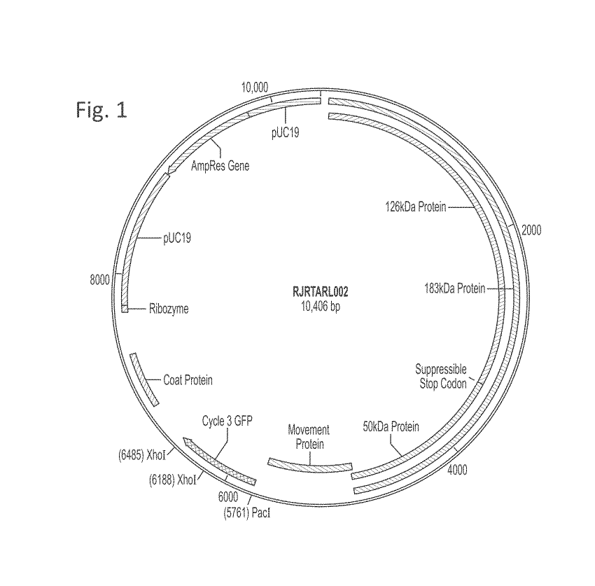

To confirm that a Geneware® vector can infect tobacco plants and produce foreign protein in tobacco cells (Pogue et al., 2010), the RJRTARL002 vector was constructed essentially as described in Example 1. FIG. 1 shows a map the RJRTARL002 vector comprising the GENEWARE® vector modified to express the cycle 3 green fluorescent protein (c3GFP). RJRTARL002 is the GENEWARE® vector having the cycle 3 Green Fluorescent Protein (c3GFP) inserted between the sequences coding for the coat protein and the movement protein (FIG. 1). The vector was used to infect tobacco (Nicotiana tabacum) K326 variety, tobacco (Nicotiana tabacum) Xanthi variety and Nicotiana benthamiana plants. Essentially, the RNA was produced from RJRTARL002 vector DNA via a commercially available transcription kit. The infective RJRTARL002 RNA was then diluted and used to inoculate plant leaves.

Transcription was performed as follows. For a reaction performed using an Ambion mMessage mMachine (Applied Biosystems/Ambion Part #: AM1344), samples included: 10 μL 2×NTP/CAP, 2 μL 10× Reaction Buffer, 1 μg vector DNA, 2 μL 10× T7 enzyme mix and nuclease free water to total volume 20 μL. Samples were then mixed and allowed to incubate at 37° C. for 2-3 hours. The reaction mix can be scaled up depending on number of leaves to be inoculated.

For inoculation, an aliquot (20 μL) of the transcript product was mixed with 804, inoculation buffer to prepare a total 1004, inoculum. Inoculation buffer was made with polished water and contains 0.75% (weight/volume) glycine, 1.05% (weight/volume) potassium phosphate dibasic (K2HPO4), 1% (weight/volume) sodium pyrophosphate decahydrate (Na4PO7.10H2O), 1% (weight/volume) bentonite and 1% (weight/volume) celite.

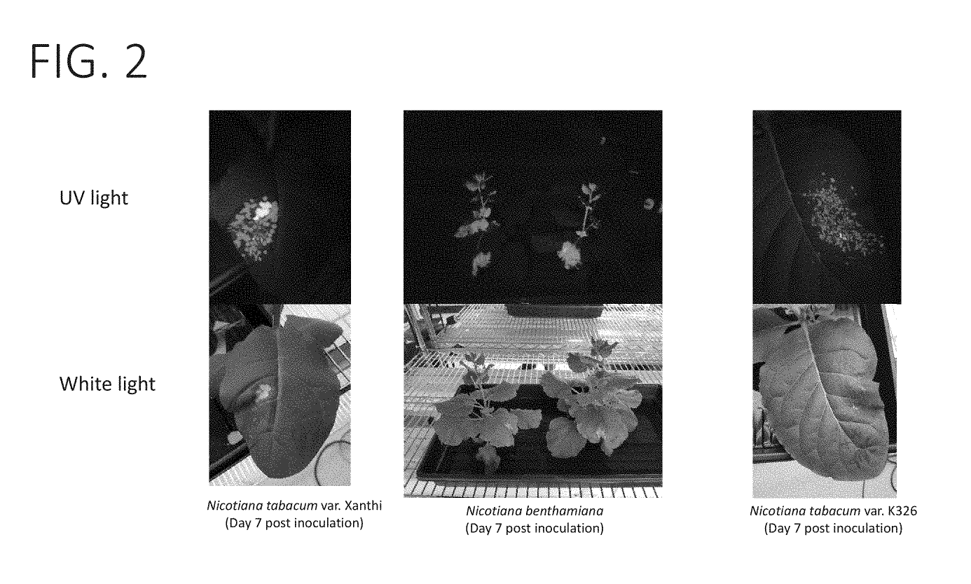

An inoculum (25 μL) was placed on each leaf and gently rubbed with a gloved finger to wound the leaf and allow entrance of virus. The inoculated plants were checked under Ultraviolet (UV) light at multiple time points after inoculation. FIG. 2 shows an example of three different types of tobacco, Nicotiana tabacum var. Xanthi, Nicotiana benthamiana, and Nicotiana tabacum var. K326 seven days post-inoculation with the RJRTARL002 vector according to an embodiment of the disclosure. The images in the top row are exposed to UV light allowing for visualization of the green fluorescent protein. The images in the bottom row are exposed to white light allowing for visualization of the area of infection. The expression of cycle 3 GFP was clearly visible. This result demonstrated that GENEWARE® vector can produce protein in tobacco plant.

B. Assessment of Next Generation Plants for Retention of TMV

Since the GENEWARE® vector is derived from tobacco virus TMV, it is possible virus infection could spread to next generation plants via infected seeds. To assess whether this occurred, seeds from two RJRTARL002 inoculated Nicotiana benthamiana plants were harvested, and approximately 40 plants germinated from these seeds were checked under ultraviolet light. No GFP was observed in these plants. Additionally, 10 randomly picked plants were tested by an Agdia ImmunoStrip® for TMV, and no TMV infection was detected in these 10 plants. These results demonstrated that no TMV infection was transmitted from GENEWARE® inoculated plants to their next generation plant via seeds.

### Example 4—Cas Based Viral Based Genome Editing Protocols to Make Clean GMO Plants

RNA-guided endonuclease Cas9 has been proven to be able to work on multiple systems including human, plant, and fungi. The methods described herein can create CRISPR-cas9 edited plant without introducing CRISPR backbone sequences to the plant genome, thereby producing a ‘clean’ GMO plant.

CRISPR-cas9 gene and gRNA sequences are transferred into the vector encoding a virus genome. Complete TMV vector transcripts are transmitted to the tobacco plants from in vitro derived transcripts. Tobacco plants are then infected using virus or multiple agrobacterium vectors containing either single Tobamovirus vector or tobamovirus and PVX vectors combined with the CRISPR/Cas system are separated into multiple vectors. Agrobacterium containing the different vectors are used to infect young plants.

With two vectors, each infect and express in the infected plant cells the components of the CRISPR/Cas system. The vectors replicate at the agrobacterium infiltration site and spread throughout the plant cells while expressing components of the CRISPR/Cas system. CRISPR and gRNA are expressed throughout the plant cells as the virus spreads and results in modification of a target site in the plant genome.

When the infected plant flowers and produces seeds, a number of genome-edited seeds are produced. Or, as an alternative, mTMV is harvested from the agrobacterium infiltration site and used to directly infect seedlings. When the seedling grows up and produces flowers and seeds, a number of genome edited seeds are produced. An embryo rescue process is performed on the seeds to obtain virus free plants from the seeds above. The resulting plant contains an edit at the target site without the trace of any foreign DNA.

Thus, the genome editing activity is only active while in the infected plant, which makes the process transient. However, when the plant grows and the modified virus spreads, more plant tissue is genome edited. Genome-edited plant seeds are obtained from this infected plant. These genome edited seeds without CRISPR sequence or other foreign DNA in the plant genome make it a non-trans genetic organism by USDA definition.

### Example 5—Model Gene Knockout

Marker genes are routinely used in plant genetic transformation protocols to ensure the selection/scoring of transformed cells/tissues from that of non-transformed. Among the selectable markers, antibiotic and herbicide resistance genes have been the most widely used in plant genetic transformation. Phytoene desaturase (PDS) is a key enzyme of carotenoid synthesis pathway and it is highly conserved—genes being characterized from a number of plant species. Matthews, et al, J. Exp. Bot., 54: 2215-2230. Loss of the catalytic activity of PDS leads to accumulation of phytoene, characterized by albino and dwarf appearance producing dwarf albino regenerants that can be used as scorable marker for genetic knockout events. Qin et al., (2007) Cell Res., 17: 471-482. RNA-guided endonuclease Cas9 has been proved to be able to work on multiple systems including human, plant, and fungi. The methods described herein create CRISPR-cas9 edited plant without introducing CRISPR backbone sequences to the plant genome, thereby producing a ‘clean’ GMO plant.

Sequences from Arabidopsis thaliana PDS gene (NM_202816.2) or PDS genes from Nicotiana tabacum, Nicotiana benthamiana, or other species can be used in the region 601-728 bp of the gene for ready disruption. CRISPR-cas9 gene and gRNA sequences are transferred into the vector encoding a virus genome. Complete TMV vector transcripts are transmitted to the tobacco plants from in vitro derived transcripts. Conversely, tobacco plants are then infected using virus or multiple Agrobacterium vectors (tobamovirus and PVX vectors) containing different virus parts. Elements of the TMV genome and components of the CRISPR/Cas system are separated into multiple vectors. Agrobacterium containing the different vectors are used to infect young plants.

Each element of the viral genome is then expressed in the infected plant cells to assemble a complete TMV modified to express components of the CRISPR/Cas system. Assembled modified virus replicates at the Agrobacterium infiltration site and spreads throughout the plant cells while expressing components of the CRISPR/Cas system. CRISPR and gRNA are expressed throughout the plant cells as the virus spreads and results in modification of a target site in the plant genome. Screening of photo-bleached tissues will allow ready identification of regions with PDS knockout phenotype.

When the infected plant flowers and produces seeds, a number of genome-edited seeds are produced—these can also be screened and selected by dwarfed, photo-bleached phenotype. Or, as an alternative, mTMV is harvested from the Agrobacterium infiltration site and used to directly infect seedlings. When the seedling grows up and produces flowers and seeds, a number of genome edited seeds are produced. An embryo rescue process is performed on the seeds to obtain virus free plants from the seeds above, and selected based on photobleaching phenotype. The resulting plant contains an edit at the target site without the trace of any foreign DNA.

Thus, the genome editing activity is only active while in the infected plant, which makes the process transient. However, when the plant grows and the modified virus spreads, more plant tissue is genome edited. Genome-edited plant seeds are obtained from this infected plant. These genome edited seeds without CRISPR sequence or other foreign DNA in the plant genome make it a non-trans genetic organism by USDA definition.

### Example 6—Preparation of a Smokeless Tobacco Composition

A smokeless tobacco composition suitable for use as a smokeless tobacco product (STP) for oral use is provided in the following manner using harvested tobacco leaves having a genome edit at the target site. A tobacco material having tobacco particles with an average particle size of about 30 microns is provided. The tobacco material is dried in open atmosphere at about 54° C. to reduce the moisture content from about 50 percent to less than about 10 percent. Various dry ingredients are provided, which include a filler (isomalt), a salt (sodium chloride), a sweetener (sucralose), and flavorants (vanillin, spray-dried peppermint, spray-dried menthol). All dry ingredients, in powder form, as well the dried tobacco material, are added together and thoroughly mixed in a Hobart mixer with a paddle for about three minutes at about 120 rpm.

A lipid substance having a melting point of about 38° C. to about 42° C. is provided. The lipid substance is a non-hydrogenated lauric coating fat containing a blend of palm kernel oil and palm oil.

The lipid substance is melted in a mixing vessel. While maintaining heat to the mixing vessel having the melted lipid substance, the mixed dry formulation is added while mixing occurs, thereby creating a flowable slurry of smokeless tobacco composition having a moisture content of less than about 10 percent. The slurry is deposited in a mold to achieve about 1 gram weight per piece of smokeless tobacco product. The slurry is allowed to harden by ambient air drying for about 45 minutes, after which the individual pieces of smokeless tobacco product are removed from the mold.

### Example 7—Embodiments of the Disclosure

A1. A method for modifying a target site in the genome of a tobacco plant cell, the method comprising introducing a RNA molecule comprising a nucleic acid sequence encoding a DNA endonuclease into the tobacco plant cell, wherein, when expressed, the DNA endonuclease introduces a modification at the target site in the genome of the tobacco plant cell.
A2. The method of embodiment A1, wherein the RNA molecule is located on a vector.
A3. The method of embodiment A2, wherein the vector is a tobacco mosaic virus (TMV) vector.
A4. The method of embodiment A3, wherein the nucleic acid sequence encoding the DNA endonuclease is operably linked to a virus coat protein promoter.
A5. The method of any one of embodiments A1-A4, further comprising synthesizing the RNA molecule comprising a nucleic acid sequence encoding a DNA endonuclease in vitro prior to introducing the RNA molecule into the plant cell.
A6. The method of embodiment A5, wherein the RNA molecule is mechanically introduced to the plant cell by rubbing, high pressure spray, or using a gene gun.
A7. The method of embodiments A5 or A6, wherein the introduced RNA molecule produces a tobacco mosaic virus expressing the DNA endonuclease.
A8. The method of any one of embodiments A2-A4, wherein the vector is introduced into the plant cell.
A9. The method of any one of embodiments A1-A8, further comprising removing a part of the plant comprising the RNA molecule and culturing the part of the plant on selection medium.
A10. The method of embodiment A9, wherein the plant part comprising the RNA molecule is removed from the leaf, meristem, shoot, and/or flower of the plant.
A11. The method of either one of embodiments A9 or A10, further comprising culturing the plant part to produce a regenerated plant.
A12. The method of any one of embodiments A9-A10, further comprising confirming the modification at the target site of the plant part.
A13. The method of any one of embodiments A1-A12 further comprising isolating at least one plant cell comprising a modification at the target site, wherein the modification includes at least one deletion, insertion, or substitution of one or more nucleotides in the target site.
A14. The method of embodiment A13, further comprising culturing a plant comprising the plant cell comprising the modification at the target site.
A15. The method of embodiment A14, wherein the plant is cultured until the plant produces seeds comprising the modification at the target site of the genome.
A16. The method of embodiment A15, further comprising performing embryo rescue on the seeds comprising the modification at the target site of the genome.
A17. The method of embodiment A16 further comprising planting seed produced from the plant comprising the modification, culturing the planted seed to produce an edited tobacco plant, harvesting the edited tobacco plant, and producing a tobacco product from the harvested plant.
A18. The method of any one of embodiments A1-A17, wherein the modification is a double strand break.
A19. The method of any one of embodiments A1-A18, wherein the DNA endonuclease is a meganuclease.
A20. The method of embodiment A19, wherein the meganuclease has been modified to be specific for the target site.
A21. The method of embodiment A20, wherein the target site is located in a gene encoding a PDS (Phytoene desaturase), nicotine synthase, or a nicotine demethylase.
A22. The method of any one of embodiments A1-A21, wherein the tobacco is a N. tabacum tobacco.
A23. The method of any one of embodiments A1-A21, wherein the tobacco is a N. rustica tobacco.
A24. Tobacco plants or plant parts produced by the method of any one of embodiments A1-A23.
A25. Tobacco seeds produced by the method of embodiment A15.
A26. A tobacco plant, tobacco plant part, or tobacco plant cell comprising a vector comprising a nucleic acid sequence encoding a DNA endonuclease.
A27. A tobacco mosaic virus (TMV) genome modified to comprise a nucleic acid sequence encoding a meganuclease operably linked to a promoter, wherein the meganuclease is specific for a target site in a gene encoding a PDS (Phytoene desaturase), nicotine synthase, or a nicotine demethylase.
A28. The TMV genome of embodiment A27, wherein the promoter is a CaMV35S, a T7 RNA polymerase promoter, or a coat protein subgenomic promoter.
A29. A vector comprising a nucleic acid sequence encoding the TMV genome of embodiment 27 or 28.
B1. A method for modifying a target site in the genome of a tobacco plant cell, the method comprising: introducing a nucleic acid encoding a functional editing component into the tobacco plant cell, wherein the functional editing component introduces a modification at the target site in the genome of the tobacco plant cell.
B2. The method of embodiment B1, wherein the functional editing component is an endonuclease that cleaves DNA.
B3. The method of embodiment B2, wherein the endonuclease is one of a meganuclease or a guide RNA and/or Cas9 endonuclease.
B4. The method of any one of embodiments B1-B3, wherein the nucleic acid comprises an RNA expression vector.
B5. The method of embodiment B4, wherein the vector is a tobacco mosaic virus (TMV) vector.
B6. The method of any one of embodiments B1-B5, wherein the functional editing component is operably linked to one of a CaMV35S, a T7 RNA polymerase promoter, or a coat protein subgenomic promoter.
B7. The method of any one of embodiments B1-B6, further comprising synthesizing the nucleic acid encoding the functional editing component in vitro prior to introducing the nucleic acid nucleic acid encoding a functional editing component into the plant cell.
B8. The method of any one of embodiments B1-B7, wherein the nucleic acid encoding the functional editing component is mechanically introduced to the plant cell by rubbing, high pressure spray, or using a gene gun.
B9. The method of any one of embodiments B1-B8, wherein the modification comprises at least one of substitution of at least one nucleotide, a deletion of at least one nucleotide, or an insertion of at least one nucleotide at the target site.
B10. The method of any one of embodiments B1-B9, further comprising removing a part of the plant comprising the nucleic acid encoding the functional editing component and culturing the part of the plant on selection medium.
B11. The method of embodiment B10, wherein the plant part comprising the nucleic acid encoding the functional editing component is removed from the leaf, meristem, shoot, and/or flower of the plant.
B12. The method of any one of embodiments B10 or B11, further comprising culturing the plant part to produce a regenerated plant.
B13. The method of any one of embodiments B1-B12, further comprising confirming the modification at the target site of the plant part.
B14. The method of any one of embodiments B1-B13 further comprising isolating at least one plant cell comprising the modification at the target site.
B15. The method of embodiment B14, further comprising culturing a plant comprising the at least one plant cell comprising the modification at the target site.
B16. The method of embodiment B15, wherein the plant is cultured until the plant produces seeds comprising the modification at the target site of the genome.
B17. The method of embodiment B16, further comprising performing embryo rescue on the seeds comprising the modification at the target site of the genome.
B18. The method of embodiment B17, further comprising planting at least one seed produced from the plant comprising the modification, culturing the planted seed to produce a tobacco plant comprising a modified target site, harvesting the edited tobacco plant, and producing a tobacco product from the harvested plant.
B19. The method of any one of embodiments B1-B18, wherein the functional editing component has been genetically engineered to be specific for the target site.
B20. The method of any one of embodiments B1-B19, wherein the target site is located in a gene encoding a PDS (Phytoene desaturase), nicotine synthase, or a nicotine demethylase.
B21. The method of any one of embodiments B1-B20, wherein the tobacco is a N. tabacum tobacco or a N. rustica tobacco.
B22. Tobacco plants or plant parts produced by the method of any one of embodiments B1-B21.
B23. Tobacco seeds produced by the method of embodiment B16.
B24. A tobacco plant, tobacco plant part, or tobacco plant cell comprising an RNA expression vector comprising a nucleic acid sequence encoding a functional editing component.
B25. The tobacco plant, tobacco plant part, or tobacco plant cell of embodiment B24, wherein the functional editing component is an endonuclease that cleaves DNA.
B26. The tobacco plant, tobacco plant part, or tobacco plant cell of any one of embodiments B24 or B25, wherein the endonuclease is one of a meganuclease or a guide RNA and/or Cas9 endonuclease.
B27. The tobacco plant, tobacco plant part, or tobacco plant cell of any one of embodiments B24-B26, wherein the RNA expression vector is a tobacco mosaic virus (TMV) vector.
B28. The tobacco plant, tobacco plant part, or tobacco plant cell of any one of embodiments B24-B26, wherein the functional editing component is operably linked to one of a CaMV35S, a T7 RNA polymerase promoter, or a coat protein subgenomic promoter.
B29. A tobacco mosaic virus (TMV) genome modified to comprise a nucleic acid sequence encoding a meganuclease operably linked to a promoter,
B30. The modified TMV genome of embodiment B29, wherein the meganuclease is specific for a target site in a gene encoding a PDS (Phytoene desaturase), nicotine synthase, or a nicotine demethylase.
B31. The modified TMV genome of embodiment B29, wherein the promoter is a CaMV35S, a T7 RNA polymerase promoter, or a coat protein subgenomic promoter.
B32. A vector comprising a nucleic acid sequence encoding the TMV genome of any one of embodiments B29-B31.

That which is claimed:

1. A method for modifying a target site in the genome of a tobacco plant cell, the method comprising: introducing a nucleic acid encoding a functional editing component into the tobacco plant cell, wherein the functional editing component introduces a modification at the target site in the genome of the tobacco plant cell.

2. The method of claim 1, wherein the functional editing component is an endonuclease that cleaves DNA.

3. The method of claim 2, wherein the endonuclease is one of a meganuclease or a guide RNA and/or Cas9 endonuclease.

4. The method of claim 1, wherein the nucleic acid encoding a functional editing component comprises an RNA expression vector.

5. The method of claim 4, wherein the vector is a tobacco mosaic virus (TMV) vector.

6. The method of claim 1, wherein the functional editing component is operably linked to one of a CaMV35S, a T7 RNA polymerase promoter, or a coat protein subgenomic promoter.

7. The method of claim 1, further comprising synthesizing the nucleic acid encoding the functional editing component in vitro prior to introducing the nucleic acid nucleic acid encoding a functional editing component into the plant cell.

8. The method of claim 1, wherein the nucleic acid encoding the functional editing component is mechanically introduced to the plant cell by rubbing, high pressure spray, or using a gene gun.

9. The method of claim 1, wherein the modification comprises at least one of substitution of at least one nucleotide, a deletion of at least one nucleotide, or an insertion of at least one nucleotide at the target site.

10. The method of claim 1, further comprising removing a part of the plant comprising the nucleic acid encoding the functional editing component and culturing the part of the plant on selection medium.

11. The method of claim 1, further comprising isolating at least one plant cell comprising the modification at the target site.

12. The method of claim 11, further comprising culturing a plant comprising the plant cell comprising the modification at the target site.

13. The method of claim 12, wherein the plant is cultured until the plant produces seeds comprising the modification at the target site of the genome.

14. The method of claim 1, wherein the functional editing component has been genetically engineered to be specific for the target site.

15. The method of claim 1, wherein the target site is located in a gene encoding a PDS (Phytoene desaturase), nicotine synthase, or a nicotine demethylase.

16. The method of claim 1, wherein the tobacco is a N. tabacum tobacco or a N. rustica tobacco.

17. A tobacco plant or plant part produced by introducing a nucleic acid encoding a functional editing component into the tobacco plant cell, wherein the functional editing component introduces a modification at the target site in the genome of the tobacco plant cell.

18. Tobacco seeds produced from the plant or plant part of claim 17.

19. A tobacco plant, tobacco plant part, or tobacco plant cell comprising an RNA expression vector comprising a nucleic acid sequence encoding a functional editing component.

20. The tobacco plant, tobacco plant part, or tobacco plant cell of claim 19, wherein the functional editing component is an endonuclease that cleaves DNA.

21. The tobacco plant, tobacco plant part, or tobacco plant cell of claim 20, wherein the endonuclease is one of a meganuclease or a guide RNA and/or Cas9 endonuclease.

22. The tobacco plant, tobacco plant part, or tobacco plant cell of claim 19, wherein the RNA expression vector is a tobacco mosaic virus (TMV) vector.

23. A tobacco mosaic virus (TMV) genome modified to comprise a nucleic acid sequence encoding a meganuclease operably linked to a promoter.

24. A vector comprising a nucleic acid sequence encoding a tobacco mosaic virus (TMV) genome modified to comprise a nucleic acid sequence encoding a meganuclease operably linked to a promoter.
